(19) United States
(12) Patent Application Publication
(10) Pub. No.: US 2023/0372346 A1
(43) Pub. Date: Nov. 23, 2023

(54) TREATMENT REGIMENS FOR EXON-20 INSERTION MUTANT EGFR CANCERS

(71) Applicant: CULLINAN PEARL CORP., Cambridge, MA (US)

(72) Inventors: Briggs MORRISON, Cambridge, MA (US); Jon WIGGINTON, Cambridge, MA (US); David WITTER, Cambridge, MA (US); Leigh ZAWEL, Cambridge, MA (US); Myles CLANCY, Cambridge, MA (US)

(73) Assignee: CULLINAN PEARL CORP., Cambridge, MA (US)

(21) Appl. No.: 18/044,236

(22) PCT Filed: Sep. 7, 2021
(86) PCT No.: PCT/US2021/049314

§ 371 Date: Mar. 7, 2023

Related U.S. Application Data
(60) Provisional application No. 63/196,562, filed on Jun. 3, 2021.
(60) Provisional application No. 63/075,810, filed on Sep. 8, 2020.

(51) Int. Cl. A61K 31/519; A61P 35/00

(52) U.S. Cl. CPC A61K 31/519; A61P 35/00

(57) ABSTRACT

Treatment regimens for exon 20 insertion mutant cancers with (S)—N-(4-amino-6-methyl-5-(quinolin-3-yl)-8,9-dihydropyrimido[5,4-b]indolizin-8-yl)acrylamide (Compound 1).

### CROSS-REFERENCE TO RELATED APPLICATIONS

This application claims the benefit of and priority to U.S. Provisional Patent Application No. 63/075,810, filed on Sep. 8, 2020 and U.S. Provisional Application No. 63/196,562, filed on Jun. 3, 2021, the disclosure of each of which is hereby incorporated by reference in its entirety for all purposes.

### BACKGROUND

Among cancer-related deaths, deaths from lung cancer are the most common in the world, and approximately 80% to 85% of lung cancers are classified as NSCLC (American Cancer Society “What Is Non-Small Cell Lung Cancer?” https://www.cancer.org/cancer/non-small-cell-lung-cancer/about/what-is-non-small-cell-lung-cancer.html. (Accessed: Jun. 6, 2018).

Somatic mutation of the epidermal growth factor receptor (EGFR), most of which are concentrated in the region of exon 18-21, is a major oncogenic driver and is present in approximately 30% to 50% and 10% to 20% of NSCLC in Asians and in Americans and Western Europeans, respectively Beau-Faller M, et al. “Rare EGFR exon 18 and exon 20 mutations in non-small-cell lung cancer on 10 117 patients: a multicentre observational study by the French ERMETIC-IFCT network”. Ann Oncol. 2014; 25:126-131; Oxnard G R, et al. “Natural history and molecular characteristics of lung cancers harboring EGFR exon 20 insertions”. J Thorac Oncol. 2013; 8:179-184; Rosell R, et al. “Screening for epidermal growth factor receptor mutations in lung cancer.” N Engl J Med. 2009:361:958-967; Kobayashi Y, Mitsudomi T. “Not all epidermal growth factor receptor mutations in lung cancer are created equal: Perspectives for individualized treatment strategy.” Cancer Sci. 2016; 107:1179-1186).

EGFR is a transmembrane glycoprotein and belongs to the ErbB family of tyrosine kinase receptors. Activating mutations in the EGFR kinase domain induce ligand-independent constitutive activation and subsequent downstream molecule phosphorylation, leading to cancer cell growth and survival (Faber A C, et al. “BIM expression in treatment-naïve cancers predicts responsiveness to kinase inhibitors.” Cancer Discov. 2011; 1:352-365; Greulich H, et al. “Oncogenic transformation by inhibitor-sensitive and -resistant EGFR mutants.” PLoS Med. 2005:2:e313).

Various tyrosine kinase inhibitors (TKIs) targeting EGFR mutations have been developed as anticancer agents: gefitinib, erlotinib, afatinib, for the primary treatment of patients with NSCLC harboring activating mutations, including exon 19 deletions and L858R, and osimertinib has been approved for treating patients with NSCLC harboring T790M acquired resistance mutations (Yang J C, et al. “Clinical activity of afatinib in patients with advanced non-small-cell lung cancer harbouring uncommon EGFR mutations: a combined post-hoc analysis of LUX-Lung 2, LUX-Lung 3, and LUX-Lung 6.” Lancet Oncol. 2015:16:830-838; Sequist L V et al. “Phase III study of afatinib or cisplatin plus pemetrexed in patients with metastatic lung adenocarcinoma with EGFR mutations.” J Clin Oncol. 2013; 31:3327-3334; Wu Y L, et al. “Afatinib versus cisplatin plus gemcitabine for first-line treatment of Asian patients with advanced non-small-cell lung cancer harbouring EGFR mutations (LUX-Lung 6): an open-label, randomised phase 3 trial.” Lancet Oncol. 2014:15:213-222; Yang J C, et al. “Afatinib versus cisplatin-based chemotherapy for EGFR mutation-positive lung adenocarcinoma (LUX-Lung 3 and LUX-Lung 6): analysis of overall survival data from two randomised, phase 3 trials.” Lancet Oncol. 2015; 16:141-151).

About 4-10% of all EGFR mutations consist of insertions in Exon 20. Compared to other EGFR mutations, the clinical response rate of NSCLC driven by EGFR exon 20 insertion mutations is much lower—patients with EGFR exon 20 insertions rarely respond to gefitinib, erlotinib or afatinib with response rates of only 8-11%. (Yang, Lancet Oncology 2015, Yasuda H, et al. “Structural, biochemical, and clinical characterization of epidermal growth factor receptor (EGFR) exon 20 insertion mutations in lung cancer.” Sci Transl Med. 2013; 5:216ra177; Wu J Y et al. “Lung cancer with epidermal growth factor receptor exon 20 mutations is associated with poor gefitinib treatment response.” Clin Cancer Res. 2008; 14:4877-4882; Yatabe Y et al. “EGFR mutation testing practices within the Asia Pacific region: results of a multicenter diagnostic survey.” J Thorac Oncol. 2015:10:438-445; Naidoo J, et al. “Epidermal growth factor receptor exon 20 insertions in advanced lung adenocarcinomas: Clinical outcomes and response to erlotinib.” Cancer. 2015; 121:3212-3220) because plasma concentrations of these drugs in clinical settings are kept low by dose limiting toxicities (DLT) caused by wild type (WT) EGFR inhibition (Yasuda H, et al. “EGFR exon 20 insertion mutations in non-small-cell lung cancer: preclinical data and clinical implications.” Lancet Oncol. 2012:13:e23-31; Eskens F A, et al. “A phase 1 dose escalation study of BIBW 2992, an irreversible dual inhibitor of epidermal growth factor receptor 1 (EGFR) and 2 (HER2) tyrosine kinase in a 2-week on, 2-week off schedule in patients with advanced solid tumours.” Br J Cancer. 2008; 98:80-85; Lacouture M E. “Mechanisms of cutaneous toxicities to EGFR inhibitors.” Nat Rev Cancer. 2006; 6:803-812). Overall survival of patients with EGFR exon 20 insertion mutations is similar to that of patients without EGFR mutant NSCLC but inferior to that of patients with EGFR exon 19 deletion or L858R advanced NSCLC. Novel targeted therapies that are safe and effective in patients with EGFR exon 20 insertions are lacking.

### SUMMARY OF THE INVENTION

In one aspect, the invention provides a method for treating a cancer characterized by the presence of one or more EGFR mutations, comprising administering to a subject in need thereof a therapeutically effective amount of (S)—N-(4-amino-6-methyl-5-(quinolin-3-yl)-8,9-dihydropyrimido[5,4-b]indolizin-8-yl)acrylamide (Compound 1) or a pharmaceutically acceptable salt thereof to thereby treat the cancer.

In some embodiments the method comprises administering to the subject a therapeutically effective amount of Compound 1 as a free base.

In some embodiments Compound 1 is orally administered.

In some embodiments, the therapeutically effective amount of Compound 1 is between about 60 mg/day and about 300 mg/day (e.g., between about 60 mg/day and about 290 mg/day, between about 60 mg/day and about 280 mg/day, between about 60 mg/day and about 270 mg/day, between about 60 mg/day and about 260 mg/day, between about 60 mg/day and about 250 mg/day, between about 60 mg/day and about 240 mg/day, between about 60 mg/day and about 230 mg/day, between about 60 mg/day and about 220 mg/day, between about 60 mg/day and about 210 mg/day).

In some embodiments the therapeutically effective amount of Compound 1 is about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day or about 300 mg/day.

In certain embodiments, the therapeutically effective amount of Compound 1 is between about 60 mg/day and about 200 mg/day (e.g., between about 60 mg/day and about 190 mg/day, between about 60 mg/day and about 180 mg/day, between about 60 mg/day and about 170 mg/day, between about 60 mg/day and about 160 mg/day, between about 20 mg/day and about 150 mg/day, between about 60 mg/day and about 140 mg/day, between about 60 mg/day and about 130 mg/day, between about 60 mg/day and about 120 mg/day, between about 60 mg/day and about 110 mg/day).

In some embodiments the therapeutically effective amount of Compound 1 is about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day or about 200 mg/day.

In certain embodiments, the therapeutically effective amount of Compound 1 is about 60 mg/day, about 90 mg/day, about 130 mg/day, about 200 mg/day or about 300 mg/day.

In some embodiments, the therapeutically effective amount of Compound 1 is about 60 mg/day.

In some embodiments, the therapeutically effective amount of Compound 1 is about 90 mg/day.

In some embodiments, the therapeutically effective amount of Compound 1 is about 130 mg/day.

In some embodiments, the therapeutically effective amount of Compound 1 is about 200 mg/day.

In some embodiments, the therapeutically effective amount of Compound 1 is about 300 mg/day.

In certain embodiments of the invention described herein, Compound 1 is administered once daily (e.g., every 24 hours).

In some embodiments, the method comprises administering to the subject once daily (e.g., every 24 hours) a dose between about 60 mg and about 300 mg (e.g., between about 60 mg and about 290 mg, between about 60 mg and about 280 mg, between about 60 mg and about 270 mg, between about 60 mg and about 260 mg, between about 60 mg and about 250 mg, between about 60 mg and about 240 mg, between about 60 mg and about 230 mg, between about 60 mg and about 220 mg, between about 60 mg and about 210 mg) of Compound 1.

In some embodiments, the method comprises administering to the subject once daily (e.g., every 24 hours) a dose of about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg or about 300 mg of Compound 1.

In some embodiments, the method comprises administering to the subject once daily (e.g., every 24 hours) a dose between about 60 mg and about 200 mg (e.g., between about 60 mg and about 190 mg, between about 60 mg and about 180 mg, between about 60 mg and about 170 mg, between about 60 mg and about 160 mg, between about 20 mg and about 150 mg, between about 60 mg and about 140 mg, between about 60 mg and about 130 mg, between about 60 mg and about 120 mg, between about 60 mg and about 110 mg) of Compound 1.

In some embodiments, the method comprises administering to the subject once daily (e.g., every 24 hours) a dose of about 60 mg, about 70 mg, about 80 mg, about 90 mg, about 100 mg, about 110 mg, about 120 mg, about 130 mg, about 140 mg, about 150 mg, between about 160 mg, about 170 mg, about 180 mg, about 190 mg or about 200 mg of Compound 1.

In some embodiments the method comprises administering to the subject once daily (e.g., every 24 hours) a dose of about 60 mg, about 90 mg, about 130 mg, about 200 mg or about 300 mg of Compound 1.

In some embodiments the method comprises administering to the subject once daily (e.g., every 24 hours) a dose of about 60 mg of Compound 1.

In some embodiments the method comprises administering to the subject once daily (e.g., every 24 hours) a dose of about 90 mg of Compound 1.

In some embodiments the method comprises administering to the subject once daily (e.g., every 24 hours) a dose of about 130 mg of Compound 1.

In some embodiments the method comprises administering to the subject once daily (e.g., every 24 hours) a dose of about 200 mg of Compound 1.

In some embodiments the method comprises administering to the subject once daily (e.g., every 24 hours) a dose of about 300 mg of Compound 1.

In some embodiments of the invention described herein, Compound 1 is administered twice daily (e.g., every 12 hours).

In some embodiments, the method comprises administering to the subject twice daily (e.g., every 12 hours) a dose between about 30 mg and about 150 mg of Compound 1 (e.g., between about 30 mg and about 145 mg, between about 30 mg and about 140 mg, between about 30 mg and about 135 mg, between about 30 mg and about 130 mg, between about 30 mg and about 125 mg, between about 30 mg and about 120 mg, between about 30 mg and about 115 mg, between about 30 mg and about 110 mg or between about 30 mg and about 105 mg).

In some embodiments, the method comprises administering to the subject twice daily (e.g., every 12 hours) a dose of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg or about 150 mg.

In some embodiments, the method comprises administering to the subject twice daily (e.g., every 12 hours) a dose between about 30 mg and about 100 mg of Compound 1 (e.g., between about 30 mg and about 95 mg, between about 30 mg and about 90 mg, between about 30 mg and about 85 mg, between about 30 mg and about 80 mg, between about 30 mg and about 75 mg, between about 30 mg and about 70 mg, between about 30 mg and about 65 mg, between about 30 mg and about 60 mg or between about 30 mg and about 55 mg).

In some embodiments, the method comprises administering to the subject twice daily (e.g., every 12 hours) a dose of about 30 mg, about 35 mg, about 40 mg, about 45 mg, about 50 mg, about 55 mg, about 60 mg, about 65 mg, about 70 mg, about 75 mg, about 80 mg, about 85 mg or about 90 mg of Compound 1.

In some embodiments, the method comprises administering to the subject twice daily (e.g., every 12 hours) a dose of about 30 mg, about 45 mg, about 65 mg, about 100 mg or about 150 mg of compound 1.

In some embodiments, the method comprises administering to the subject twice daily (e.g., every 12 hours) a dose of about 30 mg of compound 1.

In some embodiments, the method comprises administering to the subject twice daily (e.g., every 12 hours) a dose of about 45 mg of compound 1.

In some embodiments, the method comprises administering to the subject twice daily (e.g., every 12 hours) a dose of about 65 mg of compound 1.

In some embodiments, the method comprises administering to the subject twice daily (e.g., every 12 hours) a dose of about 100 mg of compound 1.

In some embodiments, the method comprises administering to the subject twice daily (e.g., every 12 hours) a dose of about 150 mg of compound 1.

In certain embodiments. Compound 1 is administered in 21-day cycles.

In some embodiments, Compound 1 is administered in consecutive 21-day cycles (e.g., without a pause between the end of one cycle and the beginning of the next cycle).

In some embodiments Compound 1 is administered until disease progression, unacceptable toxicity, or voluntary discontinuation by subject or physician.

In certain embodiments of the methods described herein, at least one (e.g., one or more) of the EGFR mutations is an exon 20 mutation.

In some embodiments, at least one (e.g., one or more) of the EGFR mutations is an exon 20 insertion mutation.

In certain embodiments, each of the EGFR exon 20 insertion mutations is independently selected from D770_N771insX, V769_D770insX, H773_V774insX, P772_H773insX, N771_P772insX, A763_Y764insX, V774_C775insX, D761_E762insX, A767_S768insX, S768_V769insX, Y764_V765insX, V765_M766insX.

In certain embodiments, each of the EGFR exon 20 insertion mutations is independently selected from A763_Y764insFQEA, A767_S768insTLA, S768_V769insVAS, S768_V769insAWT, V769_D770insGV, V769_D770insCV, V769_D770insDNV, V769_D770insGSV, V769_D770insGVV, V769_D770insMASVD, V769_D770insASV, V769_D770insGE, V769_D770delInsDGEL, D770_N771insSVD, D770_N771insNPG, D770_N771insKH, D770_N771insGNPH, D770_N771insAPW, D770_N771insD, D770_N771insDG, D770delinsGY, D770_N771insGL. D770_N771insN, D770_N771insNPH, D770_N771insSVP, D770_N771insSVQ, D770_N771insMATP, D770_N771insG, D770_N771insY, D770_N771insGF, D770_N771insGT, delD770insGY, N771_P772insH, N771_P772insN, delN771insGY, delN771insGF, N771delinsGY, N771_P772insRH, P772_H773insPR, P772_H773insYNP, P772_H773insX, P772_H773insDPH, P772_H773insDNP, P772_H773insQV, P772_H773insTPH, P772_H773insN, P772_H773insV, P772_H773insNP, P772_H773insNPH, H773_V774insH, H773_V774insNPH, H773_V774insPH, H773_V774insGNPH, H773_V774insG, H773_V774insGH, H773_V774insAH, H773_V774delInsLM, H773_V774delInsTY, V774_C775insHV.

In further embodiments, each of the EGFR exon 20 insertion mutations is independently selected from A763_Y764insFQEA, V769_D770insASV, D770_N771insSVD, D770_N771insKH, D770_N771insGNPH, V769_D770delInsDGEL, N771_P772insRH, N771delInsGY, H773_V774insPH, H773_V774insH, H773_V774insNPH, H773_V774deInsLM, V774_C775insHV.

In certain embodiments, each of the EGFR exon 20 insertion mutations is independently selected from A763_Y764insFQEA, V769_D770insASV, D770_N771insSVD, H773_V774insPH, H773_V774insH, H773_V774insNPH.

In some embodiments, each of the EGFR exon 20 insertion mutations is independently selected from V769_D770insASV, D770_N771insSVD, D770_N771insKH, D770_N771insGNPH, N771_P772insRH, N771delInsGY, H773_V774deInsLM, H773_V774insNPH, H773_V774insH, V774_C775insHV.

In some embodiments, each of the EGFR exon 20 insertion mutations is independently selected from V769_D770insASV, D770_N771insSVD, H773_V774insH, H773_V774insNPH

In some embodiments, one or more of the EGFR mutations is an exon 18 or exon 21 mutation.

In some embodiments of the invention, one or more of the EGFR mutations is an exon 18 mutation (e.g., a substitution mutation or a deletion mutation).

In some embodiments, one or more of the EGFR mutations is an exon 18 mutation selected from the group consisting of G719X (e.g., G719A, G719S or G719C) L718X (e.g., L718Q) and E709X (e.g., E709K or E709A) mutations.

In some embodiments, one or more of the EGFR mutations is an exon 21 mutation.

In some embodiments, one or more of the EGFR mutation is an exon 21 mutation selected from the group consisting of L858X (e.g., L858R) and L861X (e.g., L861Q) mutations.

In certain embodiments, one or more of the EGFR mutations is an exon 19 deletion mutation (e.g., delE746_A750 and delL747_P753insS).

In some embodiments, one or more of the EGFR mutations is an exon 20 substitution mutation.

In certain embodiments, one or more of the EGFR mutations is an exon 20 substitution mutation selected from a T790X (e.g., T790M), a L792X (e.g., L792H, L792F, L792Y) and a S768X (e.g., S768I) mutation.

In certain embodiments, one of the EGFR mutations is a T790M mutation.

In some embodiments, the cancer is characterized by an EGFR T790M mutation and an additional EGFR mutation selected from an exon 19 deletion and a L858R mutation.

In certain embodiments of the invention described herein, the cancer is selected from lung cancer, colorectal cancer, pancreatic cancer, head and neck cancers, breast cancer, ovarian cancer, uterine cancer, gastric cancer, bladder cancer, glioma or stomach cancer.

In some embodiments, the cancer is lung cancer.

In some embodiments, the cancer is non-small cell lung cancer (NSCLC).

In some embodiments, the cancer is recurrent and/or metastatic.

In some embodiments, the cancer is recurrent and/or metastatic non-small cell lung cancer (NSCLC).

In some embodiments of the invention described herein, the subject had not been previously treated with an EGFR tyrosine kinase inhibitor (e.g., gefitinib, erlotinib, afatinib, dacomitinib, osimertinib etc.)

In some embodiments, the subject had been previously treated and/or had previously responded to treatment with an EGFR tyrosine kinase inhibitor (e.g., gefitinib, erlotinib, afatinib, dacomitinib, osimertinib etc.)

In certain embodiments, the subject is not a subject who had been previously treated and/or had previously responded to treatment with an EGFR tyrosine kinase inhibitor (e.g., gefitinib, erlotinib, afatinib, dacomitinib, osimertinib, etc.

In some embodiments, the subject is resistant to treatment with an EGFR tyrosine kinase inhibitor (e.g., gefitinib, erlotinib, afatinib, dacomitinib, osimertinib, etc.).

In some embodiments, the subject had not been previously treated with an EGFR exon 20 insertion-targeting agent (e.g., poziotinib, TAK788 (mobocertinib), tarloxotinib, JNJ-61186372, etc).

In some embodiments, the subject had been previously treated with an EGFR exon 20 insertion-targeting agent (e.g., poziotinib, TAK788 (mobocertinib), tarloxotinib, JNJ-61186372, etc).

In some embodiments, the subject has been previously treated with immunotherapy (e.g., a checkpoint inhibitor).

In some embodiments, the subject had been previously treated with chemotherapy (e.g., platinum-based chemotherapy).

In other embodiments, the subject had not been previously treated with chemotherapy.

In further embodiments, the subject is treatment-naïve.

In certain embodiments, the subject is newly diagnosed.

### DETAILED DESCRIPTION OF THE INVENTION

### Brief Description of the Drawings

FIG. 1.—Plasma concentration of unbound CLN-081 in the initial 8 hours post-dosing in 3 different patients receiving 100 mg BID of CLN-081.

FIG. 2.—Area under the plasma concentration-time curve from time 0 to 8 hours of unbound CLN-081 in patients receiving increasing BID doses of CLN-081 (30 mg BID, 45 mg BID, 65 mg BID, 100 mg BID).

FIG. 3.—Maximum plasma concentration of unbound CLN-081 in patients receiving increasing BID doses of CLN-081 (30 mg BID, 45 mg BID, 65 mg BID, 100 mg BID).

FIG. 4.—Study design and patient enrollment by phase and dose as of the Apr. 1, 2021 cut-off date.

FIG. 5.—Treatment course for the 45 patients that have been dosed with CLN-081, duration of treatment, best response of the patients, as well as the current disease and treatment status of each patient.

FIG. 6.—Best response of each of 42 evaluable patients measured by the percentage volume change of sum of lesions from baseline based on scan. The prior systemic therapies received by each patient are listed on the top. Absence of a bar in the plot indicates 0% change from baseline for the patient.

FIG. 7.—Temporal dynamics of the percentage volume change of sum of lesions from baseline for each evaluable patient.

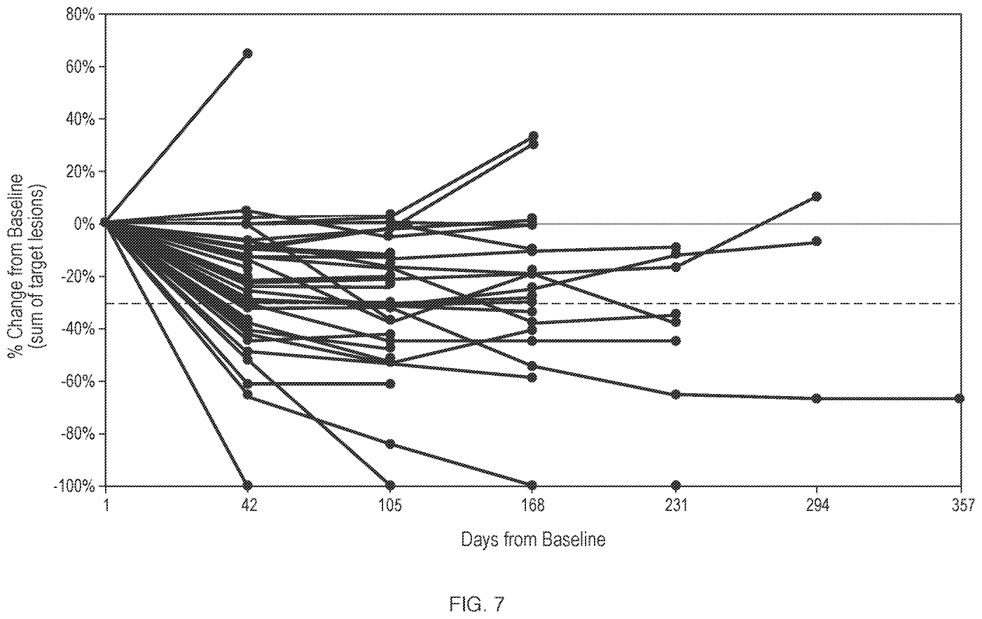

### DEFINITIONS

Unless defined otherwise, all technical and scientific terms used herein have the same meaning as commonly understood by one of ordinary skill in the art to which this invention belongs. Accordingly, the following terms are intended to have the following meanings:

As used in the specification and claims, the singular form “a”, “an” and “the” includes plural references unless the context clearly dictates otherwise.

As used herein, “antagonist” and “inhibitor” are used interchangeably, and they refer to a compound or agent having the ability to inhibit a biological function of a target protein or polypeptide, such as by inhibiting the activity or expression of the target protein or polypeptide. Accordingly, the terms “antagonist” and “inhibitor” are defined in the context of the biological role of the target protein or polypeptide. While some antagonists herein specifically interact with (e.g., bind to) the target, compounds that inhibit a biological activity of the target protein or polypeptide by interacting with other members of the signal transduction pathway of that target protein or polypeptide are also specifically included within this definition. Non-limiting examples of biological activity inhibited by an antagonist include those associated with the development, growth, or spread of a tumor, or an undesired immune response as manifested in autoimmune disease.

As used herein, “anti-cancer agent”, “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

As used herein, “cell proliferation” refers to a phenomenon by which the cell number has changed as a result of cell division. This term also encompasses cell growth by which the cell morphology has changed (e.g., increased in size) consistent with a proliferative signal.

As used herein, “administration” of a disclosed compound encompasses the delivery to a subject of a compound as described herein, or a prodrug or other pharmaceutically acceptable derivative thereof, using any suitable formulation or route of administration, as discussed herein.

As used herein, “co-administration,” “administered in combination with,” and their grammatical equivalents, as used herein, encompasses administration of two or more agents to a subject such that both agents and/or their metabolites are present in the subject at the same time. Co-administration includes simultaneous administration in separate compositions, administration at different times in separate compositions, or administration in a single fixed dose composition in which both agents are present.

As used herein, “selective inhibition” or “selectively inhibit” as applied to a biologically active agent refers to the agent's ability to selectively reduce the target signaling activity as compared to off-target signaling activity, via direct or indirect interaction with the target. For example, a compound that selectively inhibits exon 20 mutant EGFR over wild-type EGFR has an activity of at least about 2×against the mutated EGFR relative to the compound's activity against the wild-type EGFR isoform (e.g., at least about 3×, about 5×, about 10×, about 20×, about 50×, or about 100×).

As used herein, “in vivo” refers to an event that takes place in a subject's body. In vivo also includes events occurring in rodents, such as rats, mice, guinea pigs, and the like.

As used herein, “in vitro” refers to an event that takes places outside of a subject's body. For example, an in vitro assay encompasses any assay conducted outside of a subject. In vitro assays encompass cell-based assays in which cells, alive or dead, are employed. In vitro assays also encompass a cell-free assay in which no intact cells are employed.

As used herein, a “cancer characterized by the presence of one or more EGFR mutations” refers to cancer involving an aberrant EGFR-mediated signaling pathway associated with the EGFR having one or more mutations in any of its exons, including cancers having one or more mutations in the exon 20 domain. In one embodiment, the mutant EGFR has one or more mutations in the exon 20 domain. In some embodiments, the mutant EGFR-mediated disorder can be associated with EGFR having one or more mutations in the exon 20 domain.

As used herein, “therapeutic effect” encompasses a therapeutic benefit as described above. A “prophylactic effect” includes delaying or eliminating the appearance of a disease or condition, delaying or eliminating the onset of symptoms of a disease or condition, slowing, halting, or reversing the progression of a disease or condition, or any combination thereof.

As used herein, “effective amount” or “therapeutically effective amount” refers to that amount of a compound or pharmaceutical composition described herein that is sufficient to effect the intended application including, but not limited to, disease treatment, as illustrated below. In some embodiments, the amount that is effective for detectable killing or inhibition of the growth or spread of cancer cells; the size or number of tumors:or other measure of the level, stage, progression or severity of the cancer. The therapeutically effective amount can vary depending upon the intended application (in vitro or in vivo), or the subject and disease condition being treated, e.g., the weight and age of the subject, the severity of the disease condition, the manner of administration and the like. The term also applies to a dose that will induce a particular response in target cells, e.g., reduction of cell migration.

The terms “treatment”, “treating”, “palliating”, “managing” and “ameliorating” are used interchangeably herein. These terms refer to an approach for obtaining beneficial or desired results including, but not limited to, therapeutic benefit. The term “therapeutic benefit’ refers to the eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For a “prophylactic benefit,” the pharmaceutical compounds and/or compositions can be administered to a patient at risk of developing a particular disease, or to a patient reporting one or more of the physiological symptoms of a disease, even though a diagnosis of this disease may not have been made.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. In some embodiments, the subject is a human.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66: 1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

In certain embodiments, the pharmaceutically acceptable salt is a succinate salt, fumarate salt, hippurate salt, oxalate salt, mesylate salt, tosylate salt, sulfate salt, hydrochloride salt, or hydrobromide salt.

The term “pharmaceutically acceptable carrier” or “pharmaceutically acceptable excipient” includes any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. The use of such media and agents for pharmaceutically active substances is well known in the art. Except insofar as any conventional media or agent is incompatible with the active ingredient, its use in the therapeutic compositions as disclosed herein is contemplated. Non-limiting examples of pharmaceutically acceptable carriers and excipients include sugars such as lactose, glucose and sucrose: starches such as corn starch and potato starch: cellulose and its derivatives such as sodium carboxymethyl cellulose, ethyl cellulose and cellulose acetate; powdered tragacanth: malt: gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide: alginic acid; isotonic saline:Ringer's solution; ethyl alcohol:phosphate buffer solutions; non-toxic compatible lubricants such as sodium lauryl sulfate and magnesium stearate; coloring agents; releasing agents; coating agents; sweetening, flavoring and perfuming agents; preservatives; antioxidants:ion exchangers; alumina: aluminum stearate; lecithin; self emulsifying drug delivery systems (SEDDS) such as d-α-tocopherol polyethyleneglycol 1000 succinate; surfactants used in pharmaceutical dosage forms such as Tweens or other similar polymeric delivery matrices: serum proteins such as human serum albumin; glycine; sorbic acid: potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances:polyacrylates:waxes:and polyethylene-polyoxypropylene-block polymers. Cyclodextrins such as a-, b-, and g-cyclodextrin, or chemically modified derivatives such as hydroxyalkylcyclodextrins, including 2- and 3-hydroxypropyl-cyclodextrins, or other solubilized derivatives can also be used to enhance delivery of compounds described herein.

### Compound 1

In one aspect, the invention concerns (S)—N-(4-amino-6-methyl-5-(quinolin-3-yl)-8,9-dihydropyrimido[5,4-b]indolizin-8-yl)acrylamide (Compound 1) and pharmaceutically acceptable salts thereof. Compound 1 is also known as CLN-081 and TAS-6417, and has the following structure:

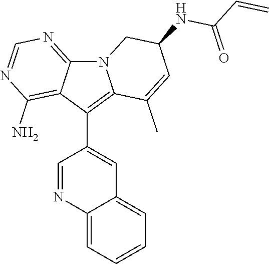 [SMILES: C=CC(=O)N[C@H]1C=C(C)c2c(-c3cnc4ccccc4c3)c3c(N)ncnc3n2C1]

The chemical synthesis and chemical properties have been described in U.S. Pat. No. 9,650,386, which is fully incorporated by reference herein.

In some embodiments the invention concerns (S)—N-(4-amino-6-methyl-5-(quinolin-3-yl)-8,9-dihydropyrimido[5,4-b]indolizin-8-yl)acrylamide (Compound 1) as the free base.

Compound 1 is a potent, and highly selective EGFR-TK1 (as described in WO2018079310, which is fully incorporated by reference herein and Hasako, S. et al “TAS6417, A Novel EGFR Inhibitor Targeting Exon 20 Insertion Mutations”, Mol Cancer Ther; 17 (8), 2018, pp. 1648-1657). Biochemical assays have shown that Compound 1 inhibited the in vitro phosphorylation activity of EGFR and EGFR mutants that harbor an exon 20 insertion mutation.

In cell-based assay. Compound 1 showed intensive cellular potency in inhibiting the phosphorylation of mutant EGFRs that possess a wide spectrum of in-frame insertion mutations in exon 20 (A763_Y764insFQEA, V769_D770insASV, D770_N771insG, D770_N771insSVD, H773_V774insNPH, and H773_V774insPH). Compound 1 also showed moderate inhibition against WT EGFR. Consistent with intracellular target inhibition, Compound 1 demonstrated a more potent and selective inhibitory effect on the proliferation of the cells expressing EGFR with exon 20 insertion mutations than on cells expressing WT EGFR.

Furthermore, Compound 1 inhibited the growth of five cell lines out of a panel of seven NSCLC cell lines harboring mutant EGFR (V769_D770insASV for LXF 2478L cells; D770_N771insSVD for NCI-H1975 EGFR D770_N771insSVD cells; delE746_A750 for HCC827 and PC-9 cells; and L858R plus T790M for NCI-H1975) with G150 values ranging from 1.92±0.21 nmol/L to 86.5±28.5 nmol/L. By contrast, KRAS mutant cell lines, NCI-H23 cells, and NCI-H460 cells that exhibit EGFR-independent cell growth did not respond to Compound 1 (GI₅₀>3000 nmol/L). CLN-081 suppressed the growth of NSCLC cells with EGFR exon 20 insertions (LXF 2478 and NCI-H1975 EGFR D770_N771insSVD cells) through a mechanism associated with the inhibition of the phosphorylation of EGFR and its downstream molecules and the induction of caspase 3/7.

In vivo antitumor efficacy studies showed that in nude mouse and/or nude rat models bearing subcutaneously-implanted tumors expressing EGFR with exon 20 insertions (V769_D770insASV, D770_N771insSVD, and H773_V774insNPH), Compound 1 exerts striking tumor growth inhibition and tumor regression effects in a dose-dependent manner. Pharmacodynamic (PD) marker analysis revealed that Compound 1 exhibits potent and durable in vivo inhibitory effect on the phosphorylation of EGFR and its downstream effectors in human lung cancer xenografts harboring EGFR exon 20 insertions but spares skin tissue with WT EGFR. The antitumor efficacy of twice-daily Compound 1 administration to nude mice transplanted with the human NSCLC cell line NCI-H1975 EGFR D770_N771insSVD is not inferior to that of once-daily Compound 1 administration.

In cell-based assay. Compound 1 showed intensive cellular potency in inhibiting the phosphorylation of mutant EGFRs that possess mutations in exon 18 or exon 21 (G719A, G719S, G719C, E709K, E709A, L861Q), and the activity was higher than erlotinib and osimertinib. Compound 1 also showed intensive inhibition against EGFRs harboring a combination of acquired resistance mutation T790M and either exon 18 or exon 21 mutations (G719A+T790M, L861Q+T790M) (WO2019045036, incorporated herein by reference in its entirety; Udagawa, H. et al. “TAS6417/CLN-081 Is a Pan-Mutation-Selective EGFR Tyrosine Kinase Inhibitor with a Broad Spectrum of Preclinical Activity against Clinically Relevant EGFR Mutations” Mol Cancer Res 2019; 17:2233-43).

Furthermore, Compound 1 inhibited the growth of Ba/F3 cell lines harboring EGFR with mutations in exon 18 (G719A, G719A+T790M) or exon 21 (L861Q, L861Q+T790M) with IC50 ranging from 9.0 nmol/L to 37.5 nmol/L, which were significantly lower than the IC50 for inhibiting Ba/F3 harboring wild type EGFR (597.3 nmol/L). The selectivity index, defined as the ratio between IC50 for WT EGFR and mutant EGFR containing cell lines, was much higher for Compound 1 compared to erlotinib and afatinib. When EGFR mutations include T790M in addition to either G719A or L861Q, the selectivity index for Compound 1 is higher than osimertinib.

In vivo antitumor efficacy studies showed that in nude mouse model bearing subcutaneously-implanted tumors expressing EGFR with G719A+T790M mutations, Compound 1 exerts significant tumor growth inhibition effects in a dose-dependent manner. Importantly, the anti-tumor activity was not effected at a cost of body weight loss, fecal abnormalities, or skin abnormalities.

In cell-based assay, Compound 1 showed intensive cellular potency in inhibiting the phosphorylation of mutant EGFRs that possess mutations in exon 18 or exon 21 (G719A, G719S, G719C, E709K, E709A, L861Q), and the activity was higher than erlotinib and osimertinib. Compound 1 also showed intensive inhibition against EGFRs harboring a combination of acquired resistance mutation T790M and either exon 18 or exon 21 mutations (G719A+T790M, L861Q+T790M).

In cell-based assay, Compound 1 showed intensive cellular potency in inhibiting the phosphorylation of mutant EGFRs that possess L718Q mutation in exon 18 in combination with ex19del+T790M or L858R+T790M (i.e., L718Q+ex19del+T790M or L718Q+L858R+T790M), and both the absolute activity and the selectivity over baseline mutations (ex19del+T790M or L858R+T790M) was higher than osimertinib, erlotinib and Afatinib (WO2020138400, incorporated herein by reference in its entirety).

In cell-based assay. Compound 1 showed intensive cellular potency in inhibiting the phosphorylation of mutant EGFRs that possess mutations at L792 (L792H, L792F, L792Y) in exon 20 in combination with ex19del+T790M or L858R+T790M (i.e. L792H+ex19del+T790M, L792H+L858R+T790M, L792F+ex19del+T790M, L792F+L858R+T790M, L792Y+ex19del+T790M, L792Y+L858R+T790M), and both the absolute activity and the selectivity over baseline mutations (ex19del+T790M or L858R+T790M) was higher than osimertinib, erlotinib and Afatinib.

### Pharmaceutical Compositions

In one aspect the invention provides Compound 1 as part of a pharmaceutical composition. In addition to Compound 1, the pharmaceutical composition can comprise a pharmaceutical carrier, thereby forming a suitable dosage form suitable for administration as described in the methods disclosed herein. Examples of dosage forms include oral preparations, injections, suppositories, ointments, patches, and the like. In one embodiment, the dosage form is an oral preparation (e.g., a tablet, coated tablet, granule, capsule, powder, etc.). Such dosage forms can be formed by methods known to persons skilled in the art.

As the pharmaceutical carrier, various conventional organic or inorganic carrier materials used as preparation materials may be blended as an excipient, binder, disintegrant, lubricant, or colorant in solid preparations, or as a solvent, solubilizing agent, suspending agent, isotonizing agent, buffer, or soothing agent in liquid preparations. Moreover, pharmaceutical preparation additives, such as antiseptics, antioxidants, colorants, sweeteners, and stabilizers, may also be used.

In certain embodiments, an oral solid preparation (e.g., an oral dosage form) comprises Compound 1 and optionally a pharmaceutically acceptable excipient. The composition can further comprise a binder, a disintegrant, a lubricant, a colorant, a taste-masking or flavoring agent, etc. In some embodiments, the oral solid preparation (i.e., the pharmaceutical composition configured for oral administration) is formulated into tablets, coated tablets, granules, powders, capsules, or the like by ordinary methods.

Examples of excipients include lactose, sucrose, D-mannitol, glucose, starch, calcium carbonate, kaolin, microcrystalline cellulose, and silicic acid anhydride. Examples of binders include water, ethanol, 1-propanol, 2-propanol, simple syrup, liquid glucose, liquid α-starch, liquid gelatin, D-mannitol, carboxymethyl cellulose, hydroxypropyl cellulose, hydroxypropyl starch, methyl cellulose, ethyl cellulose, shellac, calcium phosphate, polyvinylpyrrolidone, and the like. Examples of disintegrators include dry starch, sodium alginate, powdered agar, sodium hydrogen carbonate, calcium carbonate, sodium lauryl sulfate, stearic acid monoglyceride, lactose, and the like. Examples of lubricants include purified talc, sodium stearate, magnesium stearate, borax, polyethylene glycol, and the like. Examples of colorants include titanium oxide, iron oxide, and the like. Examples of taste-masking or flavoring agents include sucrose, bitter orange peel, citric acid, tartaric acid, and the like.

When a liquid preparation for oral administration is prepared, a taste-masking agent, a buffer, a stabilizer, a flavoring agent, and the like may be added to Compound 1; and the resulting mixture may be formulated into an oral liquid preparation, syrup, elixir, etc., according to an ordinary method.

Examples of taste-masking or flavoring agents include those mentioned above. Examples of buffer agents include sodium citrate and the like. Examples of stabilizers include tragacanth, gum arabic, gelatin, and the like. As necessary, these preparations for oral administration may be coated according to methods known in the art with an enteric coating or other coating for the purpose of, for example, persistence of effects. Examples of such coating agents include hydroxypropyl methylcellulose, ethyl cellulose, hydroxymethyl cellulose, hydroxypropyl cellulose, polyoxyethylene glycol, and Tween 80 (registered trademark).

When an injection agent is prepared, a pH regulator, a buffer, a stabilizer, an isotonizing agent, a local anesthetic, and the like, may be added to Compound 1; and the mixture may be formulated into a subcutaneous, intramuscular, or intravenous injection according to an ordinary method.

Examples of the pH adjuster and the buffer used herein include sodium citrate, sodium acetate, and sodium phosphate. Examples of the stabilizer include sodium pyrosulfite. EDTA, thioglycolic acid, and thiolactic acid. Examples of the local anesthetic include procaine hydrochloride and lidocaine hydrochloride. Examples of the tonicity agent include sodium chloride, glucose, D-mannitol, and glycerol.

When a suppository is prepared, pharmaceutically acceptable carriers known by a person skilled in the art, such as polyethylene glycol, lanolin, cacao butter, and fatty acid triglyceride; and as necessary, surfactants such as Tween 80 (registered trademark), may be added to Compound 1, and the resulting mixture may be formulated into a suppository according to an ordinary method.

When an ointment is prepared, a commonly used base, stabilizer, wetting agent, preservative, and the like, may be blended into Compound 1, as necessary:and the obtained mixture may be mixed and formulated into an ointment according to an ordinary method.

Examples of the base include liquid paraffin, white petrolatum, white beeswax, octyl dodecyl alcohol, and paraffin. Examples of the preservative include methyl paraoxybenzoate, ethyl paraoxybenzoate, and propyl paraoxybenzoate.

When a patch is prepared, the above-described ointment, cream, gel, paste, or the like, may be applied to an ordinary substrate according to an ordinary method. Examples of substrates include woven fabrics or non-woven fabrics comprising cotton, staple fibers, or chemical fibers; and films or foam sheets of soft vinyl chloride, polyethylene, polyurethane, etc., are suitable.

The amount of compound to be incorporated in each of such dosage unit forms depends on the condition of the patient to whom the compound is administered, the dosage form thereof, etc. In general, in the case of an oral agent, the amount of the compound in each dosage unit is between 5 and 200 mg (e.g., 5 mg, 10 mg, 15 mg, 20 mg, 25 mg, 30 mg, 35 mg, 40 mg, 45 mg, 50 mg, 55 mg 60 mg, 65 mg, 70 mg, 75 mg, 80 mg, 85 mg, 90 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 200 mg). In some embodiments, the amount of Compound 1 in each dosage unit is between 5 and 100 mg (e.g., 5 mg, 10 mg, 15 mg, 20 mg, 25 mg, 30 mg, 35 mg, 40 mg, 45 mg, 50 mg, 55 mg 60 mg, 65 mg, 70 mg, 75 mg, 80 mg, 85 mg, 90 mg, 100 mg). In some embodiments, the amount of Compound 1 in each dosage unit is between 5 and 50 mg (e.g., 5 mg, 10 mg, 15 mg, 20 mg, 25 mg, 30 mg, 35 mg, 40 mg, 45 mg, 50 mg).

### Methods of Treatment

In one aspect, the invention provides a method for treating a cancer characterized by the presence of one or more EGFR mutations, comprising administering to a subject in need thereof a therapeutically effective amount of (S)—N-(4-amino-6-methyl-5-(quinolin-3-yl)-8,9-dihydropyrimido[5,4-b]indolizin-8-yl)acrylamide (Compound 1) or a pharmaceutically acceptable salt thereof to thereby treat the cancer.

In certain embodiments of the methods described herein, at least one (e.g., one or more) of the EGFR mutations is an exon 20 mutation.

In some embodiments, at least one (e.g., one or more) of the EGFR mutations is an exon 20 insertion mutation.

In certain embodiments, each of the EGFR exon 20 insertion mutations is independently selected from D770_N771insX, V769_D770insX, H773_V774insX, P772_H773insX, N771_P772insX, A763_Y764insX, V774_C775insX, D761_E762insX, A767_S768insX, S768_V769insX, Y764_V765insX, V765_M766insX.

In certain embodiments, each of the EGFR exon 20 insertion mutations is independently selected from A763_Y764insFQEA, A767_S768insTLA, S768_V769insVAS, S768_V769insAWT, V769_D770insGV, V769_D770insCV, V769_D770insDNV, V769_D770insGSV, V769_D770insGVV, V769_D770insMASVD, V769_D770insASV, V769_D770insGE, V769_D770delInsDGEL, D770_N771insSVD, D770_N771insNPG, D770_N771insKH, D770_N771insGNPH, D770_N771insAPW, D770_N771insD, D770_N771insDG, D770delinsGY, D770_N771insGL, D770_N771insN, D770_N771insNPH, D770_N771insSVP, D770_N771insSVQ, D770_N771insMATP, D770_N771insG, D770_N771insY, D770_N771insGF, D770_N771insGT, delD770insGY, N771_P772insH, N771_P772insN, delN771insGY, delN771insGF, N771delinsGY, N771_P772insRH, P772_H773insPR, P772_H773insYNP, P772_H773insX, P772_H773insDPH, P772_H773insDNP, P772_H773insQV, P772_H773insTPH, P772_H773insN, P772_H773insV, P772_H773insNP, P772_H773insNPH, H773_V774insH, H773_V774insNPH, H773_V774insPH, H773_V774insGNPH, H773_V774insG, H773_V774insGH, H773_V774insAH, H773_V774delInsLM, H773_V774delInsTY, V774_C775insHV.

In further embodiments, each of the EGFR exon 20 insertion mutations is independently selected from A763_Y764insFQEA, V769_D770insASV, D770_N771insSVD, D770_N771insKH, D770_N771insGNPH, N771_P772insRH, N771delInsGY, H773_V774insPH, H773_V774insH, H773_V774insNPH, H773_V774deInsLM, V774_C775insHV.

In certain embodiments, each of the EGFR exon 20 insertion mutations is independently selected from A763_Y764insFQEA, V769_D770insASV, D770_N771insSVD, H773_V774insPH, H773_V774insH, H773_V774insNPH.

In some embodiments, each of the EGFR exon 20 insertion mutations is independently selected from V769_D770insASV, D770_N771insSVD, D770_N771insKH, D770_N771insGNPH, N771_P772insRH, N771delInsGY, H773_V774deInsLM, H773_V774insNPH, H773_V774insH, V774_C775insHV.

In some embodiments, each of the EGFR exon 20 insertion mutations is independently selected from V769_D770insASV, D770_N771insSVD, H773_V774insH, H773_V774insNPH

In some embodiments, one or more of the EGFR mutations is an exon 18 or exon 21 mutation.

In some embodiments of the invention, one or more of the EGFR mutations is an exon 18 mutation (e.g., a point mutation or a deletion mutation).

In some embodiments, one or more of the EGFR mutations is an exon 18 mutation selected from the group consisting of G719X (e.g., G719A, G719S or G719C) and E709X (e.g., E709K or E709A) mutations.

In some embodiments, one or more of the EGFR mutations is an exon 21 mutation.

In some embodiments, one or more of the EGFR mutation is an exon 21 mutation selected from the group consisting of L858X (e.g., L858R) and L861X (e.g., L861Q) mutations.

In certain embodiments, one or more of the EGFR mutations is an exon 19 deletion mutation (e.g., delE746_A750 and delL747_P753insS).

In some embodiments, one or more of the EGFR mutations is an S768I mutation.

In certain embodiments, one of the EGFR mutations is a T790M mutation.

In some embodiments, the cancer is characterized by an EGFR T790M mutation and an additional EGFR mutation selected from an exon 19 deletion and a L858R mutation.

In certain embodiments of the invention described herein, the cancer is selected from lung cancer, colorectal cancer, pancreatic cancer, head and neck cancers, breast cancer, ovarian cancer, uterine cancer, gastric cancer, bladder cancer, glioma or stomach cancer.

In some embodiments, the cancer is lung cancer.

In some embodiments, the cancer is non-small cell lung cancer (NSCLC).

In some embodiments, the cancer is head and neck cancer. In some embodiments, the cancer is sinonasal squamous cell carcinoma (SNSCC).

In some embodiments, the cancer is recurrent and/or metastatic.

In some embodiments, the cancer is recurrent and/or metastatic non-small cell lung cancer (NSCLC).

In some embodiments of the invention described herein, the subject had not been previously treated with an EGFR tyrosine kinase inhibitor (e.g., gefitinib, erlotinib, afatinib, dacomitinib, osimertinib etc.)

In some embodiments, the subject had been previously treated and/or had previously responded to treatment with an EGFR tyrosine kinase inhibitor (e.g., gefitinib, erlotinib, afatinib, dacomitinib, osimertinib etc.)

In certain embodiments, the subject is not a subject who had been previously treated and/or had previously responded to treatment with an EGFR tyrosine kinase inhibitor (e.g., gefitinib, erlotinib, afatinib, dacomitinib, osimertinib, etc.

In some embodiments, the subject is resistant to treatment with an EGFR tyrosine kinase inhibitor (e.g., gefitinib, erlotinib, afatinib, dacomitinib, osimertinib, etc.).

In some embodiments, the subject had not been previously treated with an EGFR exon 20 insertion-targeting agent (e.g., poziotinib, TAK788 (mobocertinib), tarloxotinib, JNJ-61186372, etc).

In some embodiments, the subject had been previously treated with an EGFR exon 20 insertion-targeting agent (e.g., poziotinib, TAK788 (mobocertinib), tarloxotinib, JNJ-61186372, etc).

In some embodiments, the subject had been previously treated with immunotherapy (e.g., a checkpoint inhibitor).

In some embodiments, the subject had been previously treated with chemotherapy (e.g., platinum-based chemotherapy).

In other embodiments, the subject had not been previously treated with chemotherapy.

In further embodiments, the subject is treatment-naïve.

In certain embodiments, the subject is newly diagnosed.

### Dosing Regimens

In some embodiments Compound 1 is orally administered.

In some embodiments, the therapeutically effective amount of Compound 1 is between about 60 mg/day and about 300 mg/day (e.g., between about 60 mg/day and about 290 mg/day, between about 60 mg/day and about 280 mg/day, between about 60 mg/day and about 270 mg/day, between about 60 mg/day and about 260 mg/day, between about 60 mg/day and about 250 mg/day, between about 60 mg/day and about 240 mg/day, between about 60 mg/day and about 230 mg/day, between about 60 mg/day and about 220 mg/day, between about 60 mg/day and about 210 mg/day).

In some embodiments the therapeutically effective amount of Compound 1 is about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day or about 300 mg/day.

In certain embodiments, the therapeutically effective amount of Compound 1 is between about 60 mg/day and about 200 mg/day (e.g., between about 60 mg/day and about 190 mg/day, between about 60 mg/day and about 180 mg/day, between about 60 mg/day and about 170 mg/day, between about 60 mg/day and about 160 mg/day, between about 20 mg/day and about 150 mg/day, between about 60 mg/day and about 140 mg/day, between about 60 mg/day and about 130 mg/day, between about 60 mg/day and about 120 mg/day, between about 60 mg/day and about 110 mg/day).

In certain embodiments, the therapeutically effective amount of Compound 1 is about 60 mg/day, about 90 mg/day, about 130 mg/day, about 200 mg/day or about 300 mg/day.

In some embodiments, the therapeutically effective amount of Compound 1 is about 60 mg/day.

In some embodiments, the therapeutically effective amount of Compound 1 is about 90 mg/day.

In some embodiments, the therapeutically effective amount of Compound 1 is about 130 mg/day.

In some embodiments, the therapeutically effective amount of Compound 1 is about 200 mg/day.

In some embodiments, the therapeutically effective amount of Compound 1 is about 300 mg/day.

In certain embodiments of the invention described herein. Compound 1 is administered once daily (e.g., every 24 hours).

In some embodiments, the method comprises administering to the subject once daily (e.g., every 24 hours) a dose between about 60 mg and about 300 mg (e.g., between about 60 mg and about 290 mg, between about 60 mg and about 280 mg, between about 60 mg and about 270 mg, between about 60 mg and about 260 mg, between about 60 mg and about 250 mg, between about 60 mg and about 240 mg, between about 60 mg and about 230 mg, between about 60 mg and about 220 mg, between about 60 mg and about 210 mg) of Compound 1.

In some embodiments, the method comprises administering to the subject once daily (e.g., every 24 hours) a dose of about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg or about 300 mg of Compound 1.

In some embodiments, the method comprises administering to the subject once daily (e.g., every 24 hours) a dose between about 60 mg and about 200 mg (e.g., between about 60 mg and about 190 mg, between about 60 mg and about 180 mg, between about 60 mg and about 170 mg, between about 60 mg and about 160 mg, between about 20 mg and about 150 mg, between about 60 mg and about 140 mg, between about 60 mg and about 130 mg, between about 60 mg and about 120 mg, between about 60 mg and about 110 mg) of Compound 1.

In some embodiments, the method comprises administering to the subject once daily (e.g., every 24 hours) a dose of about 60 mg, about 70 mg, about 80 mg, about 90 mg, about 100 mg, about 110 mg, about 120 mg, about 130 mg, about 140 mg, about 150 mg, between about 160 mg, about 170 mg, about 180 mg, about 190 mg or about 200 mg of Compound 1.

In some embodiments the method comprises administering to the subject once daily (e.g., every 24 hours) a dose of about 60 mg, about 90 mg, about 130 mg, about 200 mg or about 300 mg of Compound 1.

In some embodiments the method comprises administering to the subject once daily (e.g., every 24 hours) a dose of about 60 mg of Compound 1.

In some embodiments the method comprises administering to the subject once daily (e.g., every 24 hours) a dose of about 90 mg of Compound 1.

In some embodiments the method comprises administering to the subject once daily (e.g., every 24 hours) a dose of about 130 mg of Compound 1.

In some embodiments the method comprises administering to the subject once daily (e.g., every 24 hours) a dose of about 200 mg of Compound 1.

In some embodiments the method comprises administering to the subject once daily (e.g., every 24 hours) a dose of about 300 mg of Compound 1.

In some embodiments of the invention described herein, Compound 1 is administered twice daily (e.g., every 12 hours).

In some embodiments, the method comprises administering to the subject twice daily (e.g., every 12 hours) a dose between about 30 mg and about 150 mg of Compound 1 (e.g., between about 30 mg and about 145 mg, between about 30 mg and about 140 mg, between about 30 mg and about 135 mg, between about 30 mg and about 130 mg, between about 30 mg and about 125 mg, between about 30 mg and about 120 mg, between about 30 mg and about 115 mg, between about 30 mg and about 110 mg or between about 30 mg and about 105 mg).

In some embodiments, the method comprises administering to the subject twice daily (e.g., every 12 hours) a dose of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg or about 150 mg.

In some embodiments, the method comprises administering to the subject twice daily (e.g., every 12 hours) a dose between about 30 mg and about 100 mg of Compound 1 (e.g., between about 30 mg and about 95 mg, between about 30 mg and about 90 mg, between about 30 mg and about 85 mg, between about 30 mg and about 80 mg, between about 30 mg and about 75 mg, between about 30 mg and about 70 mg, between about 30 mg and about 65 mg, between about 30 mg and about 60 mg or between about 30 mg and about 55 mg).

In some embodiments, the method comprises administering to the subject twice daily (e.g., every 12 hours) a dose of about 30 mg, about 35 mg, about 40 mg, about 45 mg, about 50 mg, about 55 mg, about 60 mg, about 65 mg, about 70 mg, about 75 mg, about 80 mg, about 85 mg or about 90 mg of Compound 1.

In some embodiments, the method comprises administering to the subject twice daily (e.g., every 12 hours) a dose of about 30 mg, about 45 mg, about 65 mg, about 100 mg or about 150 mg of compound 1.

In some embodiments, the method comprises administering to the subject twice daily (e.g., every 12 hours) a dose of about 30 mg of compound 1.

In some embodiments, the method comprises administering to the subject twice daily (e.g., every 12 hours) a dose of about 45 mg of compound 1.

In some embodiments, the method comprises administering to the subject twice daily (e.g., every 12 hours) a dose of about 65 mg of compound 1.

In some embodiments, the method comprises administering to the subject twice daily (e.g., every 12 hours) a dose of about 100 mg of compound 1.

In some embodiments, the method comprises administering to the subject twice daily (e.g., every 12 hours) a dose of about 150 mg of compound 1.

In certain embodiments, Compound 1 is administered in 21-day cycles.

In some embodiments, Compound 1 is administered in consecutive 21-day cycles (e.g., without a pause between the end of one cycle and the beginning of the next cycle).

In some embodiments Compound 1 is administered until disease progression, unacceptable toxicity, or voluntary discontinuation by subject or physician.

In some embodiments, Compound 1 can be administered daily, every other day, three times a week, twice a week, weekly, bi-weekly, or another intermittent schedule. The dosing schedule can include a “drug holiday,” i.e., the drug can be administered for two weeks on, one week off, or three weeks on, one week on, or four weeks on, one week off, etc., or continuously, without a drug holiday. In some embodiments, Compound 1 is administered daily on a 28-day cycle. In other embodiments, Compound 1 is administered daily on a 21-day cycle. In some embodiments, Compound 1 is administered daily (e.g., once daily or twice daily) for at least three consecutive days, e.g., at least five consecutive days, at least seven consecutive days, at least 14 consecutive days, at least 21 consecutive days, or at least 28 consecutive days. Compound 1 may be administered orally, rectally, parenterally, intravenously, intraperitoneally, topically, transdermally, intramuscularly, subcutaneously, intracisternally, intravaginally, intranasally, sublingually, buccally, or by any other route. In some embodiments, Compound 1 is administered orally.

### Patient Selection

Patients will be selected based on the presence or lack of certain mutations as described herein in the “methods of treatment” section as determined by methods known in the art. For example, EGFR mutations, including EGFR exon 20 insertion mutations, may be demonstrated using commercially and/or clinically available tests well known to those of ordinary skill in the art. Such test can be either sequencing- or PCR-based. Non-exhaustive examples are Cobas EGFR Mutation Test v2, Therascreen EGFR RGQ PCR Kit, FoundationOne CDx, FoundationOne Liquid CDx, Guardant360 CDx, MSK-IMPACT. Other device or test validated and accepted by applicable healthcare providers and/or regulatory health authorities may also be used to test for EGFR mutations.

### EXAMPLES

### Example 1: A Phase 1/2a, Open-Label, Multi-Center Study to Assess Safety, Tolerability, Pharmacokinetics, Pharmacodynamics, and Efficacy of CLN-081 in Patients with Non-Small Cell Lung Cancer Harboring EGFR Exon 20 Insertion Mutations

### List of Terms, Acronyms, and Abbreviations

Abbreviation Definition
AE Adverse Event
ALT Alanine Transaminase (SGPT)
ALP Alkaline Phosphatase
ANC Absolute Neutrophil Count
AST Aspartate Transaminase (SGOT)
AT Accelerated Titration Design
AUC Area Under the Drug Concentration-Time Curve
BOR Best Overall Response
BP Blood Pressure
BUN Blood Urea Nitrogen
° C. Degrees Celsius
Ca Calcium
CBC w/ Diff Complete Blood Count with Differential
CFR Code of Federal Regulations
Cl Chloride
CL Clearance
Cmax Maximum Drug Concentration
Cr Creatinine
CR Complete Response
CRF Case Report Form
CSF Cerebrospinal Fluid
CT Computed tomography
CTCAE Common Toxicity Criteria for Adverse Events
DCR Disease Control Rate
DLT Dose Limiting Toxicity
DOR Duration of Response
EC Ethics Committee
ECG Electrocardiogram
ECOG Eastern Cooperative Oncology Group
eCRF Electronic Case Report Form
EGFR Epidermal Growth Factor Receptor
EMR Electronic Medical Record
EOT End of Treatment
° F. Degrees Fahrenheit
FDA Food and Drug Administration (U.S.)
GCP Good Clinical Practice
GLP Good Laboratory Practice
h or hr Hour
HED Human Equivalent Dose
HIV Human Immunodeficiency Virus
HNSTD Highest Non-Severely Toxic Dose
HR Heart Rate
IB Investigator Brochure
ICD Interstitial Lung Disease
ICF Informed Consent Form
ICH International Conference on Harmonisation
IEC Independent Ethics Committee
IND Investigational New Drug (Application)
INR International Normalised Ratio
IRB Institutional Review Board
K Potassium
kg Kilogram
L Liter
m or min Minute
m2 Meter squared
MAD Maximum Administered Dose
mg Milligram
Mg Magnesium Sulfate
mL Milliliter
MRT Mean Residence Time
ms or msec Millisecond
MTD Maximum tolerated dose
Na Sodium
NCI National Cancer Institute
NGS Next Generation Sequencing
NSCLC Non-small cell lung cancer
NYHA New York Heart Association
NYSDOH New York State Department of Health
ORR Overall Response Rate
OS Overall Survival
PFS Progression Free Survival
PD Progressive Disease or Pharmacodynamics
PH Potential Hydrogen
PI Principal Investigator
PK Pharmacokinetics
PR Partial Response
PS Performance Status
PT Prothrombin Time
PITT Partial Thromboplastin Time
QD Once Daily
R6 Rolling Six Design
QTc Corrected QT Interval
RBC Red Blood Cells
RECIST Response Evaluation Criteria in Solid Tumors
RP2D Recommended Phase 2 Dose
RR Respiratory Rate
SAE Serious Adverse Event
SAP Statistical Analysis Plan
SD Stable Disease
SOC System Organ Class
SRC Safety Review Committee
t1/2 Half-Life
tmax Time to Cmax
T Temperature
TEAEs Treatment Emergent Adverse Events
TKI Tyrosine Kinase Inhibitors
ULN Upper Limit of Normal
VS Vital Signs
WBC White Blood Cells
WOCBP Women of Child-Bearing Potential
WT Wild Type

### Overview

CLN-081 is an investigational tyrosine kinase inhibitor with potent, selective preclinical activity against activating EGFR mutations, including exon 20 insertions. A phase 1/2 first-in-human, open-label, multicenter study of CLN-081 was conducted and first results were obtained. Patients with advanced NSCLC refractory to standard therapy received twice daily oral doses (30-150 mg per dose) of CLN-081 in the dose-escalation phase.

### Clinical Trial Design

### Objectives for Phase 1

### Primary Objectives

Assess the safety and tolerability of orally administered CLN-081 monotherapy.
Define the maximum tolerated dose (MTD) of orally administered CLN-081 monotherapy.

### Secondary Objectives

To assess the anti-tumor activity of orally administered CLN-081 monotherapy.
To characterize select pharmacokinetics (PK) parameters associated with orally administered CLN-081 monotherapy.
To assess activity of orally administered CLN-081 monotherapy in patients with known central nervous system (CNS) disease.

### Exploratory Objectives

To explore both blood and intra-tumoral pharmacodynamics (PD) markers of orally administered CLN-081 monotherapy.

### Objectives for Phase 2a

### Primary Objectives

Evaluate the overall response rate (ORR) of orally administered CLN-081 monotherapy.
Define the Recommended Phase 2 dose (RP2D) of orally administered CLN-081 monotherapy.

### Secondary Objectives

Evaluate duration of response (DOR), disease control rate (DCR), median progression free survival (PFS) and overall survival (OS), and landmark PFS and OS rates of patients treated with orally administered CLN-081 monotherapy.
Confirm the safety and tolerability of orally administered CLN-081 monotherapy.
To further characterize select PK parameters associated with orally administered CLN-081 monotherapy and relationships with various measures of clinical response.
To assess activity of orally administered CLN-081 monotherapy in patients with known CNS disease.
To compare the safety, tolerability and efficacy of CLN-081 administered twice daily (BID) with the same total dose administered once daily (QD).

### Exploratory Objectives

To further characterize both blood and intra-tumoral PD markers of orally administered CLN-081 monotherapy.

### Study Design:

This is a Phase 1/2a, open-label, multicenter, first-in-human trial to evaluate the safety and tolerability, PK, PD, and preliminary efficacy of CLN-081 in patients with non-small cell lung cancer (NSCLC) harboring epidermal growth factor receptor (EGFR) exon 20 insertion mutations.

This trial is divided into three parts: Phase 1 Dose Escalation, Phase 1 Dose Expansion, and Phase 2 Dose Expansion.

### Phase 1 Dose Escalation

Dose escalation in this trial will be conducted utilizing both an Accelerated Titration coupled with a Rolling Six design.
Dose escalation will initially proceed according to the Accelerated Titration design, enrolling one new patient per dose level.
Upon any instance of a CLN-081 related Grade ≥2 toxicity during Cycle 1, dose escalation will convert to the Rolling Six design in which a total of 3-6 patients per dose level will be enrolled.

In view of any CLN-081 related Grade ≥2 AEs, dose levels originally enrolled as Accelerated Titration cohorts may further be explored by opening Rolling Six cohorts at that dose level should they demonstrate serum concentration levels associated with efficacy in preclinical models.
For Accelerated Titration cohorts: There is no restriction on prior treatment (s) with an EGFR exon 20 insertion targeting drug. There is also no requirement that patients enrolled into these cohorts have received an EGFR exon 20 insertion targeting drug.
For Rolling Six cohorts: Patients enrolled to these cohorts are required to have no prior treatment with an EGFR exon 20 insertion targeting drug.
Additionally, “Prior Exon 20” cohorts may be opened, enrolling up to six patients that have previously been treated with EGFR exon 20 insertion targeting drugs.

### Phase 1 Dose Expansion

During dose escalation, one or more cohorts may be selected for expansion, enrolling up to a total of 13 response evaluable patients across a dose level in which ≥1 objective response was observed during dose escalation, and provided that dose is deemed tolerable. The total number of response evaluable patients enrolled in a given expansion cohort will depend upon the number of response evaluable patients enrolled during dose escalation. For instance, if six response evaluable patients were enrolled at a particular dose level during escalation, an additional seven will be enrolled in that dose level's expansion.

### Phase 2a Dose Expansion

Further dose expansion in the Phase 2a part of the trial may be explored. Up to an additional 23 response evaluable patients may be enrolled at any dose level deemed tolerable in which ≥4 confirmed objective responses in the group of 13 response evaluable patients originally enrolled as part of that dose level's Phase 1 escalation and expansion. Additionally, one or more cohorts may be added in which patients are dosed QD in addition to the initial BID dosing cohorts.

For each patient, the trial will consist of three periods:
Screening: Up to 28 days prior to the initiation of treatment.
Treatment: CLN-081 will be dosed QD and/or BID during each 21-day cycle.
Follow-Up: Patients will be followed for safety after study drug discontinuation and survival after entering long-term follow-up.

### Dose Escalation Procedures:

The starting dose for CLN-081 is 30 mg BID.

Each dose escalation cohort will consist of either:
1 dose limiting toxicity (DLT) evaluable patient (Accelerated Titration cohorts), or
3-6 DLT evaluable patients (Rolling Six cohorts).

To be considered evaluable for DLT, a patient will meet one of the following criteria:
Received ≥67% of CLN-081 doses during Cycle 1, or,
Received at least one dose of CLN-081 and experienced a DLT during Cycle 1.

### For Accelerated Titration Cohorts

Upon completion of an accelerated titration cohort, if:
No CLN-081 related Grade ≥2 AEs occur during Cycle 1; dose escalation may proceed to the next accelerated titration cohort (no greater than a 100% dose increase).
The patient experiences any CLN-081 related Grade ≥2 AE during Cycle 1 (not meeting the definition of a DLT); convert the current cohort to the Rolling Six design and enroll an additional 2-6 patients.
The patient experiences a DLT: convert to the Rolling Six design and enroll an additional 2-6 patients.

### For Rolling Six Cohorts

Upon completion of a Rolling Six cohort (a minimum of three, maximum of six patients), if:
0 of 3 patients experience a DLT; dose escalation may proceed to the next cohort (no greater than a 50% dose increase)
1 of 3 patients experience a DLT; an additional three evaluable patients will be enrolled into the cohort
≤1 of 6 patients experience a DLT; dose escalation may proceed to the next cohort (no greater than a 50% dose increase)
≥2 patients experience a DLT; the MTD has been exceeded and further enrollment into that cohort will cease. Lower-dose cohorts may then be explored until the MTD has been determined, including intermediate doses

Intra-patient dose escalation may be allowed during Phase 1 Dose Escalation on a case-by-case basis after agreement between the investigator and Sponsor and based upon the criteria below.

### Intra-Patient Dose Escalation in Accelerated Titration Cohorts

After an accelerated titration cohort has been declared safe by the SRC, patients at lower dose levels may be considered for intra-patient dose escalation if they meet the following criteria:
Have stable disease (SD) or better after Cycle 2 (or later)
Experienced no CLN-081 related Grade 2 AEs during the previous cycle
Have no other safety concerns as assessed by the investigator

The escalated dose must be agreed upon by the Investigator and Sponsor and may proceed beginning on Day 0 of the next cycle. Any patient that has their dose escalated will not be evaluated for DLT assessment at their new dose.

### Intra-Patient Dose Escalation in Rolling Six Cohorts

After a Rolling Six cohort has enrolled a minimum of three patients and has been declared safe by the SRC, patients at lower dose levels may then be considered for intra-patient dose escalation if they meet the following criteria:
Have stable disease (SD) or better after Cycle 2 (or later)
There are no safety concerns specific to escalating the patient's dose, as assessed by the investigator

The escalated dose must be agreed upon by the Investigator and Medical Monitor and may proceed beginning on Day 1 of the next cycle. Any patient that has their dose escalated will not be evaluated for DLT assessment at their new dose.

### Definition of DLT

DLTs will be determined based on the incidence and intensity of AEs related to CLN-081 (excluding toxicities clearly related to disease progression or other inter-current illness) as defined below. Dose escalation decisions will be determined based on DLT that occur during the 21 days after initiation of CLN-081 dosing. AEs meeting the definition of a DLT that are observed after 21 days will be made available to the SRC in case a pattern of late toxicity emerges. Toxicity will be graded by the Common Toxicity Criteria for Adverse Events (CTCAE) v5.0. Patients who experience AEs meeting the criteria for DLT should pause treatment with CLN-081 pending management and resolution of the event, and assessment of the relationship of the AE to CLN-081 administration.

Hematologic and Non-Hematologic DLT will be defined as outlined below:

### Hematologic Toxicity

Grade 4 neutropenia >7 days
Grade 4 thrombocytopenia or Grade 3 thrombocytopenia associated with bleeding or requiring platelet transfusion
Grade 3 febrile neutropenia
Grade 4 anemia

### Non-Hematologic Toxicity

Non-hematological Grade 3-4 events

The following events are excluded: Grade 3 fatigue, asthenia, fever, anorexia, constipation that last ≤7 days; Grade 3 nausea, vomiting, or diarrhea or mucositis that has resolved to Grade ≤2 within 48 hours; Grade 3 or 4 isolated electrolyte abnormalities that last ≤72 hours; Grade 3 rash that lasts ≤7 days Other
Any Grade 5 event
Any AE not otherwise meeting the criteria of dose-limiting, but is declared a DLT by the SRC

### Definition of the MTD

The MTD is defined as the highest dose level of CLN-081 at which fewer than two DLTs are observed in six evaluable dose escalation patients.

### Definition of the RP2D

The RP2D may be the MTD, the maximum administered dose (MAD) if no MTD is defined, or another dose less than the MTD, based on cumulative review of safety, efficacy, PK and PD data.

### Definition of a Response Evaluable Patient:

For the purposes of evaluating a dose level for expansion, a patient will be response evaluable if they meet the prior treatment criteria for the Rolling Six, Phase 1 Expansion, and Phase 2a Expansion cohorts and have one of the following:
At least one post-dose tumor assessment,
Discontinued prior to the first efficacy assessment due to clinical disease progression or toxicity,
Died either on treatment or within 2 days of last CLN-081 dose.

### Initiation of Phase 1 Expansion Cohorts

The SRC may elect to initiate a Phase 1 Dose Expansion Cohort and enroll an additional seven patients at any dose level that is at or below the MTD if ≥1 patient has an objective response per RECIST v1.1 (i.e., a Partial Response (PR) or Complete Response (CR)) and the cohort of existing patients has been deemed tolerable by the SRC. Confirmation of response is not required.

Based upon the above criteria, the SRC may elect to explore multiple dose levels as Phase 1 Expansion Cohorts.

### Initiation of Phase 2a Expansion Cohorts

Upon completion of any Phase 1 Expansion Cohort, the SRC may elect to expand the cohort further, initiating a Phase 2a Dose Expansion and enrolling an additional 23 response evaluable patients if the dose level has met the following:
≥4 patients have confirmed objective responses per RECIST v1.1 (i.e., a PR or CR).
The cohort of existing patients has been deemed tolerable by the SRC.

Upon completion of a Phase 2a Expansion cohort, the total number of response evaluable patients across a given dose level will be up to 36 (e.g., 6 Rolling Six patients, 7 from the Phase 1 expansion, and 23 from the Phase 2a expansion).

Based upon the above criteria, the SRC may elect to explore multiple dose levels as Phase 2a Expansion Cohorts.

### Initiation of OD Dosing

If one or more dose levels progress to Phase 2a Dose Expansion based upon the pre-specified efficacy criteria and supported by clinical safety data, the SRC may elect to initiate additional cohorts at these dose levels exploring CLN-081 on a QD schedule in contrast to the previously studied BID dosing.

In these situations, the total daily dose of CLN-081 administered QD will be the same as the corresponding BID cohort's total daily dose. For example, if a 115 mg BID cohort met the criteria to advance to the Phase 2a Dose Expansion, a proposed 230 mg QD cohort could also be initiated with the intent of enrolling a total of 36 patients-provided 230 mg BID had already been explored in the Phase 1 Dose Escalation phase of the trial and found to not exceed the MTD.

Using this same example, if 230 mg BID had not been explored in the Phase 1 Dose Escalation part of the trial, one or more Rolling Six cohorts would first be enrolled following the Phase 1 Dose Escalation specified study procedures in order to establish clinical safety. The 230 mg dose may be the starting dose, or an intermediate dose level between the highest dose explored in the BID dose escalation phase and the proposed 230 mg QD dose could first be explored. Enrollment would follow the Phase 1 Dose Escalation specified study procedures, with subsequent expansion to a total of 36 patients following the Phase 2a Dose Expansion specified study procedures based upon observed clinical safety.

### Study Population

### Inclusion Criteria

A patient who meets all of the following inclusion criteria will be eligible to participate in this study.
Histologically or cytologically confirmed recurrent and/or metastatic NSCLC.
Documented EGFR exon 20 insertion mutation demonstrated by a test routinely used by each institution and performed in a CLIA-certified or equivalent laboratory.
Prior treatment in the recurrent/metastatic disease setting including:
   a. A platinum-based chemotherapy regimen (or other chemotherapy regimen if platinum-based chemotherapy is contra-indicated)
   b. Any other approved standard therapy that is available to the patient, unless this therapy is contraindicated, intolerable to the patient, or is declined by the patient. In the case of a patient declining such therapy, documentation that the patient has been informed and declined should be documented in the medical record.
Measurable disease by RECIST 1.1.
Age ≥18 years.
Eastern Cooperative Oncology Group (ECOG) performance status 0 or 1.
Ability to take pills by mouth.
Have the following laboratory values:
   a. Serum creatinine <1.5×ULN or if higher than normal range, calculated creatinine clearance (CrCl) must be ≥50 mL/min/1.73 m² (by Cockroft-Gault formula):actual body weight must be used for CrCl unless BMI >30 kg/m² then lean body weight must be used.
   b. Total bilirubin ≤1.5×ULN unless prior history of Gilbert's syndrome.
   c. Aspartate transaminase and alanine transaminase ≤2.5×ULN, or ≤5×ULN if due to liver involvement by tumor.
   d. Hemoglobin ≥9.0 g/dL in the absence of transfusion ≤14 days prior to first dose of study drug on C1D1.
   e. Platelets ≥100×109 cells/L.
   f. Absolute neutrophil count ≥1.5×109 cells/L.
Ability to understand and the willingness to sign a written informed consent document and comply with study procedures.

### Exclusion Criteria

A patient who meets any of the following exclusion criteria will be ineligible to participate in this study:

### Prior Exon 20 Patients Only

No Prior treatment with an EGFR exon 20 insertion-targeting drug (e.g., poziotinib, TAK788 (mobocertinib), tarloxotinib, JNJ-61186372).

### Rolling Six, Phase 1 Expansion, and Phase 2a Expansion Patients Only

Prior treatment with an EGFR exon 20 insertion-targeting drug (e.g., poziotinib, TAK788 (mobocertinib), tarloxotinib, JNJ-61186372).

### All Patients

Treatment with any of the following:
   a. An EGFR tyrosine kinase inhibitors (TKIs)≤8 days or 5× the terminal phase elimination half-lives, whichever is longer, prior to the first dose of study drug on C1D1
   b. Systemic anticancer treatment (excluding EGFR-TKIs as described above)≤14 days prior to the first dose of study drug on C1D1.
   c. Radiotherapy ≤28 days and palliative radiation ≤14 days prior to the first dose of study drug on C1D1. If irradiated, lesions must have demonstrated clear-cut progression prior to being eligible for evaluation as target lesions.
   d. Immunotherapy ≤28 days prior to the first dose of study drug on C1D1.
   e. Major surgery (excluding placement of vascular access)≤28 days of the first dose of study drug on C1D1.
Have any unresolved toxicity of Grade ≥2 from previous anti-cancer treatment, except for alopecia and skin pigmentation. Patients with chronic, but stable Grade 2 toxicities may be allowed to enroll after agreement between the Investigator and Sponsor.
Have known or suspected brain metastases or spinal cord compression, unless the condition has been asymptomatic, treated with surgery and/or radiation, and has been stable without requiring escalating corticosteroids or anti-convulsant medications for at least four weeks prior to the first dose of study drug on C1D1.
Prior therapy with CLN-081.
Known hypersensitivity to CLN-081 or any drugs similar in structure or class.
Past medical history of interstitial lung disease, treatment-related pneumonitis which required steroid treatment, or any evidence of clinically active interstitial lung disease.
Cardiac conditions as follows: Patient has a history of congestive heart failure (CHF) Class III/IV according to the New York Heart Association (NYHA) Functional Classification or serious cardiac arrhythmias requiring treatment.
Resting corrected QT interval (QTc) >470 msec.
Patient is unable to take drugs orally due to disorders or diseases that may affect gastrointestinal function, including but not limited to inflammatory bowel diseases (e.g., Crohn's disease, ulcerative colitis) or malabsorption syndrome, or procedures that may affect gastrointestinal function, such as gastrectomy, enterectomy, or colectomy.
Have any condition or illness that, in the opinion of the investigator, might compromise patient safety or interfere with the evaluation of the safety of the drug.
Pregnant or lactating women; women of child-bearing potential (WOCBP) must have a negative serum pregnancy test ≤7 days prior to receiving study drug on C1D1. WOCBP and males with partners of child-bearing potential must agree to use adequate birth control (Appendix 3) throughout their participation and for six months following the last dose of study treatment.
History of another primary malignancy ≤2 years prior to starting study drug on C1D1, except for adequately treated basal or squamous cell carcinoma of the skin or cancer of the cervix in situ.
Uncontrolled intercurrent illness including, but not limited to, uncompensated respiratory, cardiac, hepatic, or renal disease, active infection (including HIV and active clinical tuberculosis), or renal transplant; ongoing or active infection, symptomatic congestive heart failure, unstable angina pectoris, cardiac arrhythmia, active peptic ulcer disease or gastritis, or psychiatric illness/social situations that would limit compliance with study requirements.
For patients with a history of hepatitis B, active infection as defined by a positive HBsAg test and detectable HBV DNA. Patients ineligible due to detectable levels of HBV DNA at baseline may be rescreened for enrollment if their HBV DNA levels become undetectable after treatment with antiviral agents, and upon agreement between the investigator and Sponsor.
For patients with a history of hepatitis C, active infection as defined by a reactive HCV antibody test and detectable HCV RNA.
Active bleeding disorders.
Is, in the Investigator's opinion, unable or unwilling to comply with the trial procedures.

### Safety Evaluation

Safety and tolerability will be assessed by the incidence and severity of AEs as determined by CTCAE v5.

### Efficacy Evaluation

The antitumor activity of study treatment will be assessed according to RECIST v1.1 guideline

### Results

### Patient Characteristics

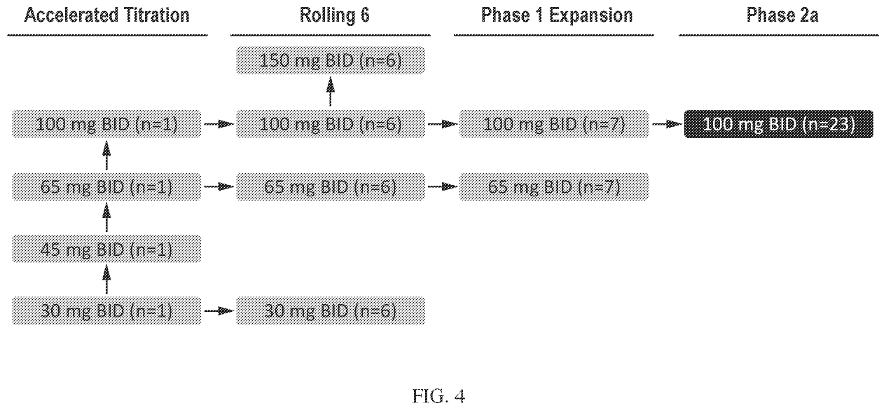

As part of this phase 1/2 study, dose levels including 30, 45, 65, 100 and 150 mg BID were explored during the escalation phases. Efficacy expansions were initiated at 30, 65 and 100 mg BID. A Phase 2a expansion was initiated at 100 mg BID after meeting protocol specified safety and efficacy criteria. A summary of the enrolment by dose and phase is shown in FIG. 4. Tumor assessments were performed at baseline, after 6 weeks of CLN-081 administration and every 9 weeks thereafter.

As of the Apr. 1, 2021 cut-off date, the following numbers of patients were enrolled in total at each of the 5 dose levels: 30 mg BID (n=7), 45 mg BID (n=1), 65 mg BID (n=14), 100 mg BID (n=37), and 150 mg BID (n=6).

As of the cut-off date 45 patients had received at least one dose of CLN-081. 42 of the 45 patients were response evaluable; one patient discontinued treatment before their first post-baseline response assessment, and two patients had not been restaged at the time of the data cut-off, but remained on treatment.

The patients were identified to have EGFR Ins 20 mutations by local. CLIA-certified or equivalent testing. All patients had received ≥1 prior systemic platinum chemotherapy regimen; 32/44 (73%) patients received ≥2 prior therapies before study entry; 25/44 (56%) patients received prior immunotherapy and 18/44 (40%) received a prior EGFR TKI.

Table 1 shows the characteristics of the patients enrolled in the clinical trial up to the aforementioned cut-off date, including demographics and information about prior therapies

TABLE 1

| Characteristic | All patients (n = 44) | |
|---|---|---|
| Median age, years (range) | 64 | (44-82) |
| Male/Female/Not Reported, n (%) | 20 (47)/22 (51)/1 (2) | |
| Race, n (%) | | |
| Asian | 15 | (35) |
| Black | 2 | (5) |
| White | 24 | (56) |
| Other or not reported | 2 | (5) |
| Stable, asymptomatic brain mets at baseline, n (%) | 12 | (27) |
| ECOG 0/1, n (%) | 15 (34)/29 (66) | |
| Number of prior systemic therapies, Median, (range) | 2 | (1-9) |
| 1, n (%) | 12 | (27) |
| 2, n (%) | 17 | (39) |
| ≥3, n (%) | 15 | (34) |
| Prior afatinib or gefitinib, n (%) | 8 | (18)) |
| Prior Osimertinib, n (%) | 9 | (20) |
| Prior poziotinib and/or TAK788 (mobocertinib), | 4 | (9) |
| n (%) | | |
| Prior immunotherapy, n (%) | 25 | (56) |

### Pharmacokinetics

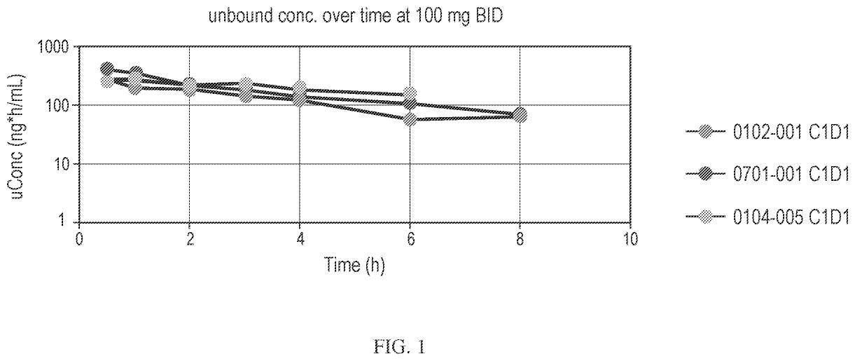

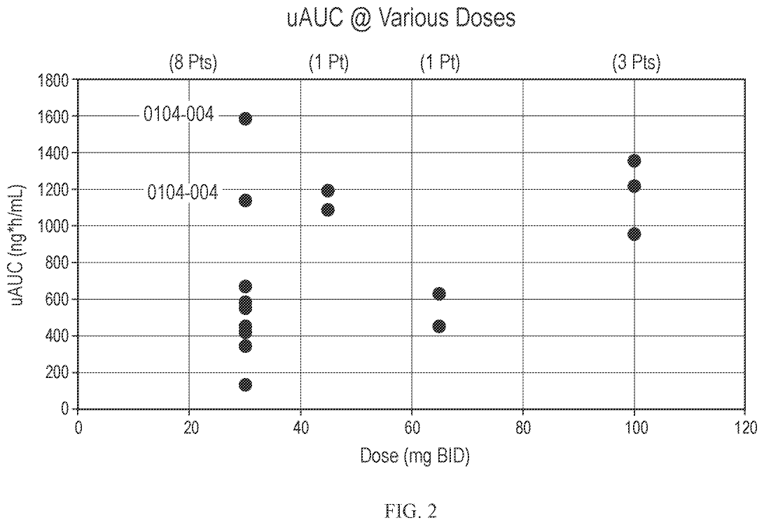

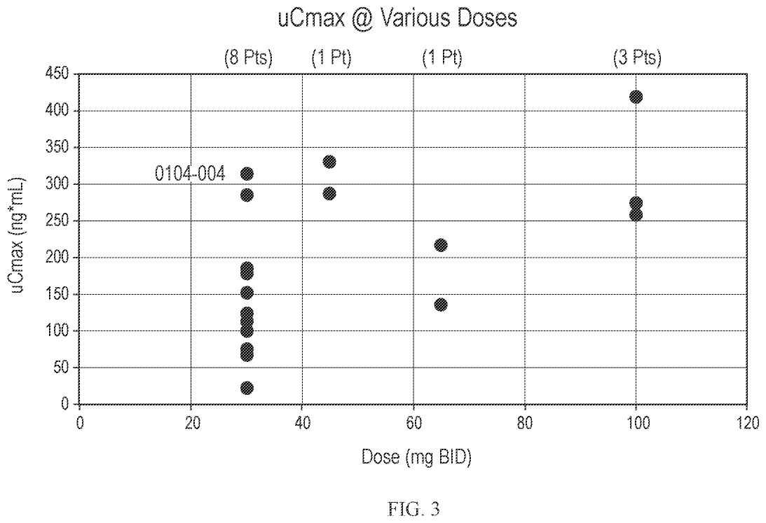

PK analysis has been performed with CLN-081 plasma concentrations determined at pre-dose, 0.5, 1, 2, 3, 4, 6, 8 and 24 hour % post initial dose on Cycle (C) I Day (D) I and C1D15. Representative temporal dynamics of unbound plasma concentrations in three different patients receiving 100 mg BID CLN-081 are shown in FIG. 1; the temporal profiles are generally comparable between the three patients. The corresponding PK parameters of these three patients are listed in Table 2. As shown in FIG. 2, area under the plasma concentration-time curve from time 0 to 8 hours (AUC_{0-8 hr}) values have been observed to generally increase with increasing dose from 30 to 100 mg CLN-081 BID. As shown in FIG. 3, the maximum plasma concentration (Cₘₐₓ) values have been observed to also generally increase with increasing dose from 30 to 100 mg CLN-081 BID. Minimal to no increase has been observed in systemic exposure levels from C1D1 to C1D15. CLN-081 has typically been rapidly absorbed with tₘₐₓ values ranging from 0.5 to 2 hours and a mean average terminal phase elimination half-life (t_{1/2}) of approximately 4 hours.

TABLE 2

| Patient | uAUC* (ng*h/mL) | uCₘₐₓ (ng/mL) | T_{1/2} (h) |
|---|---|---|---|
| 0102-001 | 957 | 260 | 3.6 |
| 0104-005 | 1213 | 274 | 6.1 |
| 0701-001 | 1353 | 418 | 3.6 |

### Safety and Adverse Events

As of the cut-off date, 44/45 (98%) of the patients experienced an adverse event (AE), irrespective of grade or attribution, including 20/45 (44%) of patients which experienced grade (Gr) ≥3 events. The most common AEs, regardless of attribution of grade, have been rash, anemia and diarrhea.

As of the cut-off date, 44/45 (98%) of patients experienced a treatment related adverse event ((TRAE), irrespective of grade, including 8/45 (18%) of patients which experienced Gr ≥3 events. One patient experienced a dose limiting toxicity (DLT), with Gr 3 diarrhea at 150 mg BID. Dose reductions were required in 5 (11%) patients. Treatment-related discontinuations were required in 4 (9%) patients.

In terms of wild-type EGFR associated TRAEs, no patients experienced Gr ≥3 treatment-related rash, only one patient experienced treatment-related Gr 3 diarrhea, and one patient discontinued CLN-081 due to treatment-related Gr 2 pneumonitis; this patient had also experienced pneumonitis while on osimertinib. A list of TRAEs experienced by more than 15% of patients and of Gr ≥3 TRAEs regardless of frequency is shown in Table 3. A more detailed breakdown of the TRAEs of highest interest by dose and grade is shown in Table 4.

TABLE 3

| TRAEs in ≥15% of patients | | | Grade ≥3 TRAEs in all patients | |
|---|---|---|---|---|
| (N = 45) | | | (N = 45) | |
| Preferred Term | n (%) | | Preferred Term | n (%) |
| Rash | 34 | (76) | Anemia | 4 (9) |
| Diarrhea | 10 | (22) | Increased AST | 2 (4) |
| Paronychia | 10 | (22) | Increased ALT | 2 (4) |
| Stomatitis | 8 | (18) | Diarrhea | 1 (2) |
| Nausea | 8 | (18) | Increased Amylase | 1 (2) |
| Anemia | 8 | (18) | Neutropenia | 1 (2) |
| Increased AST | 7 | (16) | Stomatitis | 1 (2) |
| Dry Skin | 7 | (16) | | |

TABLE 4

| | Dose (BID) | | | | |
|---|---|---|---|---|---|
| Safety population, n | 30 mg | 45 mg | 65 mg | 100 mg | 150 mg |
| DLTs, n | — | — | — | — | 1 |

Grade 1 TRAEs

| | | | | | |
|---|---|---|---|---|---|
| Rash, n | 6 | — | 7 | 5 | 4 |
| Diarrhea, n | 2 | — | 1 | 3 | 1 |
| Elevated ALT, n | — | — | 1 | 1 | 1 |
| Elevated AST, n | 1 | — | 1 | 1 | 1 |
| Anemia, n | — | — | 1 | 2 | — |

Grade 2 TRAEs

| | | | | | |
|---|---|---|---|---|---|
| Rash, n | — | — | 6 | 5 | 1 |
| Diarrhea, n | — | — | — | 1 | 1 |
| Elevated ALT, n | — | — | — | — | — |
| Elevated AST, n | — | — | — | 1 | — |
| Anemia, n | — | — | — | 1 | — |

Grade 3 TRAEs

| | | | | | |
|---|---|---|---|---|---|
| Rash, n | — | — | — | — | — |
| Diarrhea, n | — | — | — | — | 1 |
| Elevated ALT, n | — | — | 1 | 1 | — |
| Elevated AST, n | — | — | 1 | — | 1 |
| Anemia, n | 1 | — | 2 | — | 1 |

Grade 4 TRAEs

| | | | | | |
|---|---|---|---|---|---|
| Rash, n | — | — | — | — | — |
| Diarrhea, n | — | — | — | — | — |
| Elevated ALT, n | — | — | — | — | 1 |
| Elevated AST, n | — | — | — | — | — |
| Anemia, n | — | — | — | — | — |

### Efficacy

Of the 42 evaluable patients across dose levels, objective responses (all partial responses (PR)) were observed in 21 patients (50%). Of the 21 patients achieving an objective response, 13 were confirmed and 8 were unconfirmed, including 5 patients pending a confirmatory scan as of the data cut-off. At 100 mg BID, 7/13 (54%) evaluable patients achieved a PR, including 6 confirmed and 1 unconfirmed. Across dose levels, 41/42 (98%) of patients achieved stable disease (SD) or PR as the best response. Only one patient had disease progression as the best response. Disease control (PR or SD ≥6 months) was achieved in 27/42 (64%) of patients across all doses.

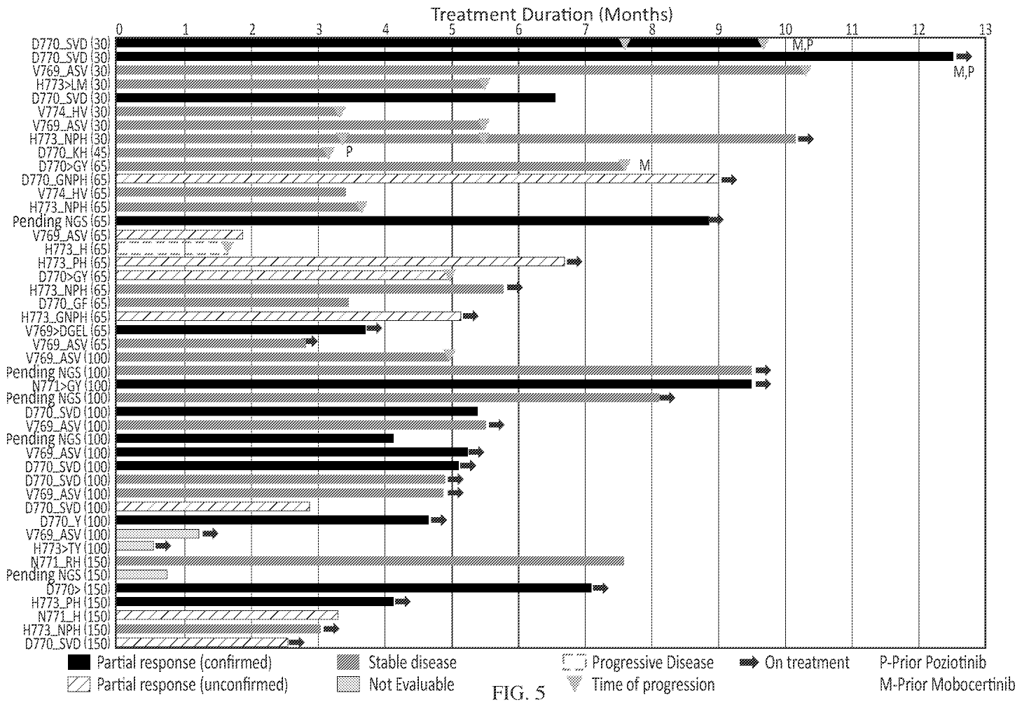

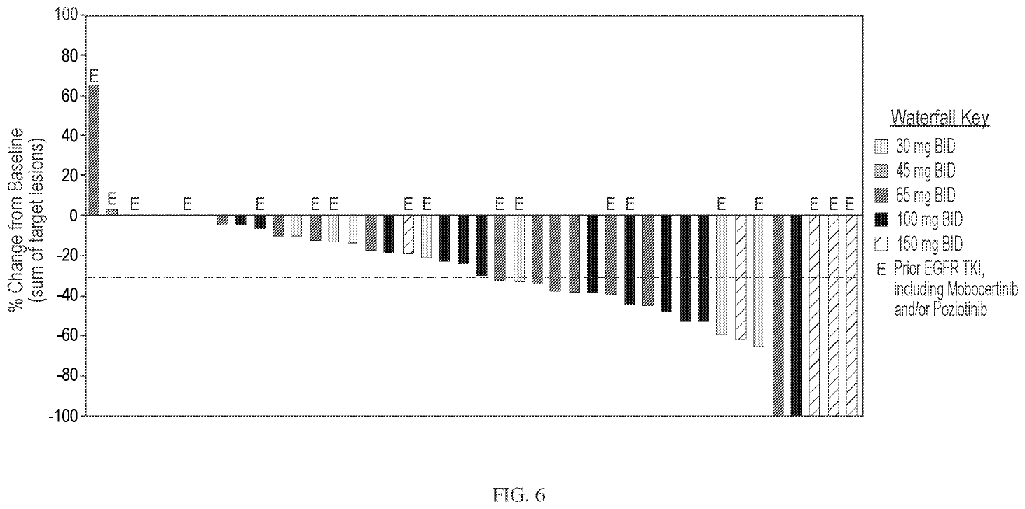

As shown in FIG. 5 and FIG. 6, partial response started to be reported at the 30 mg BID cohort, the lowest dose cohort in our dose escalation portion of the trial.

Disease assessments for patients who had at least one disease assessment following treatment with CLN-081 are shown in Table 5. A visual representation of the responses and duration of treatment for each patient is shown in FIG. 5. At the data cut-off, 22/42 (52)% of the response-evaluable patients across dose levels remained on treatment. A waterfall plot depicting the changes in target lesions by dose and previous treatment is shown in FIG. 6. All patients who responded had previously been treated with at least one of platinum-based chemotherapy, and a proportion of the patients had been previously treated with EGFR TKIs, or PD-1 inhibitors. Specifically, some of the patients who showed partial responses had been treated with one or more other EGFR TKIs targeting EGFR exon 20 insertion mutations, namely poziotinib and TAK788 (mobocertinib) (FIG. 5). It was surprising that patients who failed to respond to at least one other EGFR exon 20 insertion targeted TKI would respond to CLN-081.

TABLE 5

| | 30 mg | 45 mg | 65 mg | | 100 mg | | 150 mg |
|---|---|---|---|---|---|---|---|
| Best response, n | BID | BID | BID | | BID | | BID |
| (%) | (n = 8) | (n = 1) | (n = 14) | | (n = 13) | | (n = 6) |
| PR | 3 (38) | 0 | 7 | (50) | 7 | (54) | 4 (67) |
| SD | 5 (62) | 1 (100) | 6 | (43) | 6 | (46) | 2 (33) |
| PD | 0 | 0 | 1 | (7) | 0 | | 0 |
| Confirmed response | 3 (38) | 0 | 2 | (14) | 6 | (46) | 2 (33) |
| Unconfirmed | 0 | 0 | 2 | (14) | 1 | (8) | 0 |
| response | | | | | | | |
| Pending | 0 | 0 | 3 | (21) | 0 | | 2 (33) |
| confirmation | | | | | | | |
| Disease Control | 5 (62) | 0 | 8 | (57) | 9 | (69) | 5 (83) |
| Rate | | | | | | | |
| (PR + SD ≥ 6 mo) | | | | | | | |

### Summary

CLN-081 has an acceptable safety profile, including reduced frequency and severity of diarrhea compared to historical experience with other EGFR inhibitors. To date, AEs have been manageable and reversible, with no need for prophylaxis of GI or skin-related toxicity. The pharmacokinetic profile shows a dose proportional increase in Cₘₐₓ and AUC, with no evidence of meaningful drug accumulation across the dose range tested.

CLN-081 has encouraging antitumor activity in this heavily-pretreated patient population, including activity across a spectrum of EGFR Ins 20 variants and across a range of dose levels. CLN-081 shows high rates of response with encouraging disease control, including in patients that had progressed on prior EGFR TKIs, including poziotinib and/or TAK788 (mobocertinib), and patients that progressed on prior treatments with immunotherapies (e.g., checkpoint inhibitors). CLN-081 showed superior safety profile at efficacious dose levels compared to other TKIs targeting EGFR exon 20 insertion mutations. For example, in the clinical trial of TAK-788 (mobocertinib), the lowest-dose cohort where stable diseases or partial responses were observed also presented both grade 3 or above TRAEs and dose-limiting toxicities. On the other hand, for CLN-081, partial responses were registered in all dose cohorts tested so far, and only one instance of dose limiting toxicity was recorded to date, in the highest dose cohort tested.

1. A method for treating a cancer characterized by the presence of one or more EGFR mutations, comprising administering to a subject in need thereof a therapeutically effective amount of (S)—N-(4-amino-6-methyl-5-(quinolin-3-yl)-8,9-dihydropyrimido[5,4-b]indolizin-8-yl)acrylamide (Compound 1) or a pharmaceutically acceptable salt thereof to thereby treat the cancer.

2. The method of claim 1 wherein the method comprises administering to the subject a therapeutically effective amount of Compound 1 as a free base.

3. The method of claim 1 or 2 wherein Compound 1 is orally administered.

4. The method of any one of claims 1 to 3 wherein the therapeutically effective amount of Compound 1 is between about 60 mg/day and about 300 mg/day (e.g., between about 60 mg/day and about 290 mg/day, between about 60 mg/day and about 280 mg/day, between about 60 mg/day and about 270 mg/day, between about 60 mg/day and about 260 mg/day, between about 60 mg/day and about 250 mg/day, between about 60 mg/day and about 240 mg/day, between about 60 mg/day and about 230 mg/day, between about 60 mg/day and about 220 mg/day, between about 60 mg/day and about 210 mg/day).

5. The method of claim 4 wherein the therapeutically effective amount of Compound 1 is about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day or about 300 mg/day.

6. The method of claim 1 wherein the therapeutically effective amount of Compound 1 is between about 60 mg/day and about 200 mg/day (e.g., between about 60 mg/day and about 190 mg/day, between about 60 mg/day and about 180 mg/day, between about 60 mg/day and about 170 mg/day, between about 60 mg/day and about 160 mg/day, between about 20 mg/day and about 150 mg/day, between about 60 mg/day and about 140 mg/day, between about 60 mg/day and about 130 mg/day, between about 60 mg/day and about 120 mg/day, between about 60 mg/day and about 110 mg/day).

7. The method of claim 6 wherein the therapeutically effective amount of Compound 1 is about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, between about 160 mg/day, about 170 mg/day, about 180 mg/day, about 190 mg/day or about 200 mg/day.

8. The method of any one of claims 1 to 3 wherein the therapeutically effective amount of Compound 1 is about 60 mg/day, about 90 mg/day, about 130 mg/day, about 200 mg/day or about 300 mg/day.

9. The method of any one of claims 1 to 3 wherein the therapeutically effective amount of Compound 1 is about 60 mg/day.

10. The method of any one of claims 1 to 3 wherein the therapeutically effective amount of Compound 1 is about 90 mg/day.

11. The method of any one of claims 1 to 3 wherein the therapeutically effective amount of Compound 1 is about 130 mg/day.

12. The method of any one of claims 1 to 3 wherein the therapeutically effective amount of Compound 1 is about 200 mg/day.

13. The method of any one of claims 1 to 3 wherein the therapeutically effective amount of Compound 1 is about 300 mg/day.

14. The method of any one of claims 1 to 13 wherein Compound 1 is administered once daily (e.g., every 24 hours).

15. The method of any one of claims 1 to 3 comprising administering to the subject once daily (e.g., every 24 hours) a dose between about 60 mg and about 300 mg (e.g., between about 60 mg and about 290 mg, between about 60 mg and about 280 mg, between about 60 mg and about 270 mg, between about 60 mg and about 260 mg, between about 60 mg and about 250 mg, between about 60 mg and about 240 mg, between about 60 mg and about 230 mg, between about 60 mg and about 220 mg, between about 60 mg and about 210 mg) of Compound 1.

16. The method of claim 15 comprising administering to the subject once daily (e.g., every 24 hours) a dose of about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg or about 300 mg of Compound 1.

17. The method of claim 1 comprising administering to the subject once daily (e.g., every 24 hours) a dose between about 60 mg and about 200 mg (e.g., between about 60 mg and about 190 mg, between about 60 mg and about 180 mg, between about 60 mg and about 170 mg, between about 60 mg and about 160 mg, between about 20 mg and about 150 mg, between about 60 mg and about 140 mg, between about 60 mg and about 130 mg, between about 60 mg and about 120 mg, between about 60 mg and about 110 mg) of Compound 1.

18. The method of claim 17 comprising administering to the subject once daily (e.g., every 24 hours) a dose of about 60 mg, about 70 mg, about 80 mg, about 90 mg, about 100 mg, about 110 mg, about 120 mg, about 130 mg, about 140 mg, about 150 mg, between about 160 mg, about 170 mg, about 180 mg, about 190 mg or about 200 mg of Compound 1.

19. The method of any one of claims 1 to 3 comprising administering to the subject once daily (e.g., every 24 hours) a dose of about 60 mg, about 90 mg, about 130 mg, about 200 mg or about 300 mg of Compound 1.

20. The method of any one of claims 1 to 3 comprising administering to the subject once daily (e.g., every 24 hours) a dose of about 60 mg of Compound 1.

21. The method of any one of claims 1 to 3 comprising administering to the subject once daily (e.g., every 24 hours) a dose of about 90 mg of Compound 1.

22. The method of any one of claims 1 to 3 comprising administering to the subject once daily (e.g., every 24 hours) a dose of about 130 mg of Compound 1.

23. The method of any one of claims 1 to 3 comprising administering to the subject once daily (e.g., every 24 hours) a dose of about 200 mg of Compound 1.

24. The method of any one of claims 1 to 3 comprising administering to the subject once daily (e.g., every 24 hours) a dose of about 300 mg of Compound 1.

25. The method of any one of claims 1 to 13 wherein Compound 1 is administered twice daily (e.g., every 12 hours).

26. The method of any one of claims 1 to 3 comprising administering to the subject twice daily (e.g., every 12 hours) a dose between about 30 mg and about 150 mg of Compound 1 (e.g., between about 30 mg and about 145 mg, between about 30 mg and about 140 mg, between about 30 mg and about 135 mg, between about 30 mg and about 130 mg, between about 30 mg and about 125 mg, between about 30 mg and about 120 mg, between about 30 mg and about 115 mg, between about 30 mg and about 110 mg or between about 30 mg and about 105 mg).

27. The method of claim 26 comprising administering to the subject twice daily (e.g., every 12 hours) a dose of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg or about 150 mg.

28. The method of any one of claims 1 to 3 comprising administering to the subject twice daily (e.g., every 12 hours) a dose between about 30 mg and about 100 mg of Compound 1 (e.g., between about 30 mg and about 95 mg, between about 30 mg and about 90 mg, between about 30 mg and about 85 mg, between about 30 mg and about 80 mg, between about 30 mg and about 75 mg, between about 30 mg and about 70 mg, between about 30 mg and about 65 mg, between about 30 mg and about 60 mg or between about 30 mg and about 55 mg).

29. The method of claim 27 comprising administering to the subject twice daily (e.g., every 12 hours) a dose of about 30 mg, about 35 mg, about 40 mg, about 45 mg, about 50 mg, about 55 mg, about 60 mg, about 65 mg, about 70 mg, about 75 mg, about 80 mg, about 85 mg or about 90 mg of Compound 1.

30. The method of claim 27 comprising administering to the subject twice daily (e.g., every 12 hours) a dose of about 30 mg, about 45 mg, about 65 mg, about 100 mg or about 150 mg of compound 1.

31. The method of claim 27 comprising administering to the subject twice daily (e.g., every 12 hours) a dose of about 30 mg of compound 1.

32. The method of claim 27 comprising administering to the subject twice daily (e.g., every 12 hours) a dose of about 45 mg of compound 1.

33. The method of claim 27 comprising administering to the subject twice daily (e.g., every 12 hours) a dose of about 65 mg of compound 1.

34. The method of claim 27 comprising administering to the subject twice daily (e.g., every 12 hours) a dose of about 100 mg of compound 1.

35. The method of claim 27 comprising administering to the subject twice daily (e.g., every 12 hours) a dose of about 150 mg of compound 1.

36. The method of any one of claims 1 to 35 wherein Compound 1 is administered in 21-day cycles.

37. The method of any one of claims 1 to 36 wherein Compound 1 is administered in consecutive 21-day cycles (e.g., without a pause between the end of one cycle and the beginning of the next cycle).

38. The method of any one of claims 1 to 37 wherein Compound 1 is administered until disease progression, unacceptable toxicity, or voluntary discontinuation by subject or physician.

39. The method of any one of claims 1 to 38 wherein at least one (e.g., one or more) of the EGFR mutations is an exon 20 mutation.

40. The method of any one of claims 1 to 39 wherein at least one (e.g., one or more) of the EGFR mutations is an exon 20 insertion mutation.

41. The method of claim 40 wherein each of the EGFR exon 20 insertion mutations is independently selected from D770_N771insX, V769_D770insX, H773_V774insX, P772_H773insX, N771_P772insX, A763_Y764insX, V774_C775insX, D761_E762insX, A767_S768insX, S768_V769insX, Y764_V765insX, V765_M766insX.

42. The method of claim 40 or 41 wherein each of the EGFR exon 20 insertion mutations is independently selected from A763_Y764insFQEA, A767_S768insTLA, S768_V769insVAS, S768_V769insAWT, V769_D770insGV, V769_D770insCV, V769_D770insDNV, V769_D770insGSV, V769_D770insGVV, V769_D770insMASVD, V769_D770insASV, V769_D770insGE, V769_D770delInsDGEL, D770_N771insSVD, D770_N771insNPG, D770_N771insKH, D770_N771insGNPH, D770_N771insAPW, D770_N771insD, D770_N771insDG, D770delinsGY, D770_N771insGL, D770_N771insN, D770_N771insNPH, D770_N771insSVP, D770_N771insSVQ, D770_N771insMATP, D770_N771insG, D770_N771insY, D770_N771insGF, D770_N771insGT, delD770insGY, N771_P772insH, N771_P772insN, delN771insGY, delN771insGF, N771delinsGY, N771_P772insRH, P772_H773insPR, P772_H773insYNP, P772_H773insX, P772_H773insDPH, P772_H773insDNP, P772_H773insQV, P772_H773insTPH, P772_H773insN, P772_H773insV, P772_H773insNP, P772_H773insNPH, H773_V774insH, H773_V774insNPH, H773_V774insPH, H773_V774insGNPH, H773_V774insG, H773_V774insGH, H773_V774insAH, H773_V774delInsLM, H773_V774delInsTY, V774_C775insHV.

43. The method of any one of claims 40 to 42 wherein each of the EGFR exon 20 insertion mutations is independently selected from A763_Y764insFQEA, V769_D770insASV, D770_N771insSVD, D770_N771insKH, D770_N771insGNPH, N771_P772insRH, N771delInsGY, H773_V774insPH, H773_V774insH, H773_V774insNPH, H773_V774deInsLM, V774_C775insHV

44. The method of any one of claims 40 to 42 wherein each of the EGFR exon 20 insertion mutations is independently selected from A763_Y764insFQEA, V769_D770insASV, D770_N771insSVD, H773_V774insPH, H773_V774insH, H773_V774insNPH.

45. The method of any one of claims 40 to 42 wherein each of the EGFR exon 20 insertion mutations is independently selected from V769_D770insASV, D770_N771insSVD, D770_N771insKH, D770_N771insGNPH, N771_P772insRH, N771delInsGY, H773_V774deInsLM, H773_V774insNPH, H773_V774insH, V774_C775insHV

46. The method of any one of claims 40 to 42 wherein each of the EGFR exon 20 insertion mutations is independently selected from V769_D770insASV, D770_N771insSVD, H773_V774insH, H773_V774insNPH

47. The method of any one of claims 1 to 46 wherein one or more of the EGFR mutations is an exon 18 or exon 21 mutation.

48. The method of claim 47 wherein one or more of the EGFR mutations is an exon 18 mutation (e.g., a point/substitution mutation or a deletion mutation).

49. The method of claim 47 or 48 wherein one or more of the EGFR mutations is an exon 18 mutation selected from the group consisting of G719X (e.g., G719A, G719S or 0719C), L718X (e.g., L718Q) and E709X (e.g., E709K or E709A) mutations.

50. The method of claim 47 wherein one or more of the EGFR mutations is an exon 21 mutation.

51. The method of claim 47 or 50 wherein one or more of the EGFR mutation is an exon 21 mutation selected from the group consisting of L858X (e.g., L858R) and L861X (e.g., L861Q) mutations.

52. The method of any one of claims 1 to 51 wherein one or more of the EGFR mutations is an exon 19 deletion mutation (e.g., delE746_A750 and delL747_P753insS).

53. The method of any one of claims 1 to 52 wherein one or more of the EGFR mutations is an exon 20 point/substitution mutation.

54. The method of claim 53 wherein one or more of the EGFR mutations is an exon 20 point mutation/substitution mutation selected from a T790X (e.g., T790M), a L792X (e.g., L792H, L792F, L792Y) and a S768X (e.g., S768I) mutation.

55. The method of any one of claims 1 to 54 wherein one of the EGFR mutations is a T790M mutation.

56. The method of any one of claims 1 to 55 wherein the cancer is characterized by an EGFR T790M mutation and an additional EGFR mutation selected from an exon 19 deletion and a L858R mutation.

57. The method of any one of claims 1 to 56 wherein the cancer is selected from lung cancer, colorectal cancer, pancreatic cancer, head and neck caners, breast cancer, ovarian cancer, uterine cancer, gastric cancer, bladder cancer, glioma or stomach cancer.

58. The method of claim 57 wherein the cancer is lung cancer.

59. The method of claim 58 wherein the cancer is non-small cell lung cancer (NSCLC).

60. The method of any one of claims 1 to 59 wherein the cancer is recurrent and/or metastatic.

61. The method of any one of claims 1 to 60 wherein the cancer is recurrent and/or metastatic non-small cell lung cancer (NSCLC).

62. The method of any one of claims 1 to 61 wherein the subject had not been previously treated with an EGFR tyrosine kinase inhibitor (e.g., gefitinib, erlotinib, afatinib, dacomitinib, osimertinib, etc.)

63. The method of any one of claims 1 to 61 wherein the subject had been previously treated and/or had previously responded to treatment with an EGFR tyrosine kinase inhibitor (e.g., osimertinib, gefitinib, erlotinib, afatinib, dacomitinib, etc.)

64. The method of any one of claims 1 to 61 wherein the subject is not a subject who had been previously treated and/or had previously responded to treatment with an EGFR tyrosine kinase inhibitor (e.g., gefitinib, erlotinib, afatinib, dacomitinib, osimertinib, etc).

65. The method of any one of claims 1 to 61 wherein the subject is resistant to treatment with an EGFR tyrosine kinase inhibitor (e.g., gefitinib, erlotinib, afatinib, dacomitinib, osimertinib, etc.)

66. The method of any one of claims 1 to 61 wherein the subject had not been previously treated with an EGFR exon 20 insertion-targeting agent (e.g., poziotinib, TAK788 (mobocertinib), tarloxotinib, JNJ-61186372, etc).

67. The method of any one of claims 1 to 61 wherein the subject had been previously treated with an EGFR exon 20 insertion-targeting agent (e.g., poziotinib, TAK788 (mobocertinib), tarloxotinib, JNJ-61186372, etc).

68. The method of any one of claims 1 to 67 wherein the subject had been previously treated with immunotherapy (e.g., a checkpoint inhibitor) The method of any one of claims 1 to 68 wherein the subject had been previously treated with chemotherapy (e.g., platinum-based chemotherapy).

69. The method of any one of claims 1 to 68 wherein the subject had not been previously treated with chemotherapy.

70. The method of any one of claims 1 to 61 wherein the subject is treatment-naïve.

71. The method of any one of claims 1 to 61 wherein the subject is newly diagnosed.
